(19) United States
(12) Patent Application Publication
(10) Pub. No.: US 2008/0311145 A1
(43) Pub. Date: Dec. 18, 2008

(54) PROTEIN CAGE IMMUNOTHERAPEUTICS

(75) Inventors: Brian K. Campion, Burlingame, CA (US); Marvin I. Siegel, Blue Bell, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SpeciGen, Inc., Burlingame, CA (US)

(21) Appl. No.: 12/062,427

(22) Filed: Apr. 3, 2008

Related U.S. Application Data
(60) Provisional application No. 60/910,117, filed on Apr. 4, 2007.

(51) Int. Cl. A61K 39/02; A61K 39/12; A61P 35/00

(52) U.S. Cl. 424/190.1; 424/185.1

(57) ABSTRACT

The present invention provides compositions of heat shock protein cages for use in therapeutic vaccines. The heat shock protein cages of the invention have attached antigen, located either on the interior or exterior of the protein cage, and optionally an adjuvant.

### CROSS-REFERENCES TO RELATED APPLICATIONS

The present application claims priority to U.S. Ser. No. 60/910,117, filed Apr. 4, 2007, herein incorporated by reference.

### BACKGROUND OF THE INVENTION

Vaccination is a form of immunotherapy that has been used with remarkable success against infectious diseases such as smallpox, polio, measles, rubella, mumps, and shingles, among others. In the area of infectious diseases, success has been obtained by using vaccines composed of living, weakened strains of viruses or by using killed or inactivated organisms or purified products derived from them. Four types of vaccines are commonly used in infectious diseases: (1) Inactivated—these are previously virulent microorganisms that have been killed with chemicals or heat. Examples are vaccines against flu, cholera, bubonic plague, and hepatitis A; (2) Live, attenuated—these are live microorganisms that have been cultivated under conditions that disable their virulent properties. They typically provoke more durable immunological responses and are the preferred type for healthy adults. Examples include yellow fever, measles, rubella, and mumps; (3) Toxoids—these are inactivated toxic compounds from microorganisms in cases where these (rather than the micro-organism itself) cause illness. Examples of toxoid-based vaccines include tetanus and diphtheria; (4) Subunit—rather than introducing a whole inactivated or attenuated micro-organism to an immune system, a fragment of it can create an immune response. Characteristic examples include the subunit vaccine against HBV that is composed of only the surface proteins of the virus (produced in yeast) and the virus like particle (VLP) vaccine against Human Papillomavirus (HPV) that is composed of the viral major capsid protein.

As described above, the vast majority of vaccines developed to date are prophylactic (e.g., to prevent or ameliorate the effects of a future infection by any naturally occurring pathogen), as opposed to therapeutic (e.g., administered after the onset of a disease, such as cancer). A current goal in the area of immunotherapeutics is to adapt the remarkable success of vaccines for infectious diseases to other types of diseases. One area of active research is in the area of cancer vaccines. However, the ability to develop effective cancer vaccines has been hampered by a number of features of cancer cells, including: (1) cancers may be composed of heterogeneous cells (i.e., a tumor can have many different types of cells in it, each with different phenotypes such as having a varied assortment of cell-surface antigens); (2) cancer cells are endogenously derived within an individual with cancer, and therefore they display few antigens that would be recognized by the immune system as being foreign to that individual; (3) cancer cells may evolve mechanisms that prevent efficient recognition by the hosts' immune system. Factors such as these make it more difficult for the immune system to distinguish cancer cells from normal cells. Some progress has been made in the area of cancer immunotherapy with the discovery that many kinds of tumor cells display unusual antigens that are either inappropriate for the cell type and/or its environment, or which are only normally present during the organisms' development (e.g., fetal antigens). One example of such an antigen is the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies. Other kinds of tumor cells display cell surface receptors that are rare or absent on the surfaces of healthy cells, and which are responsible for activating cellular signal transduction pathways that cause the unregulated growth and division of the tumor cell. Examples include ErbB2, a constitutively active cell surface receptor that is produced at abnormally high levels on the surface of breast cancer tumor cells. As a result of such discoveries, a handful of monoclonal antibody therapies are available, including Alemtuzumab, Bevacizumab, Cetuximab, Gemtuzumab ozogamicin, Rituximab, and Trastuzumab, which recognize different cell surface proteins on cancer cells. It is well known that viruses that transform normal cells into dysplastic tissue and cancer produce oncogenic proteins and peptides from these proteins which are detectable on the diseased cells. One example is the human papilloma virus which is associated with genital warts, cervical and anal dysplasia and cervical and anal cancer. Cells transformed by this viral family express antigens derived from the virus. While some progress has been made, more effective and general approaches toward the development of immunotherapies against diseases such as cancer or HIV infection, which have been resistant to treatment by such approaches, is needed. The present invention satisfies these and other needs.

### BRIEF SUMMARY OF THE INVENTION

The present invention provides compositions of heat shock protein cages for use in therapeutic vaccines. As discussed in detail herein, the heat shock protein cages of the invention have attached antigen and optionally an adjuvant, either of which can be located either on the interior or exterior of the protein cage or both. Alternatively, the heat shock protein cages of the invention can be formulated in a preparation which contains the adjuvant.

In one embodiment, the present invention provides a therapeutic vaccine which comprises a heat shock protein assembled into a protein cage, having an interior and exterior, and an antigen. In various aspects of this embodiment, the heat shock protein can be derived from a variety of sources including a bacterium, a mycobacterium, a yeast, a plant, and an animal. In one advantageous aspect, a heat shock protein from an Archaebacterium such as Methannococcus jannaschii, Mycobacterium tuberculosis, Thermococcus, and Sulfolobus tokodaii may be used in the practice of this invention. Examples of Archaebacterial heat shock proteins include: Methannococcus jannaschii Hsp 16.5, Mycobacterium tuberculosis Acr1, Thermococcus sp. strain KS-1 sHsp, Sulfolobus tokodaii strain 7 StHsp 19.7, and Sulfolobus tokodaii strain 7 StHsp 14.0. Examples of heat shock proteins from other species that may be used in the practice of this invention include: wheat Hsp 16.9, Saccharomyces pompi spHsp 16.0, Saccharomyces cerevisiae Hsp27, and human Hsp27.

In various aspects of the invention, the protein cage comprises a tertiary structure of multiple subunits that form the protein cage. One such tertiary structure comprises a spherical oligomer, which may have a diameter in the range of 2 to 100 nanometers and multiple subunits that number 12, 16, or 24 subunits. In some aspects, the multiple subunits are modified as compared to the wild type protein. Examples of such modifications include subunits modified to comprise one or more cysteine or lysine residues.

In different aspects of this embodiment, the antigen can be on the interior or exterior of the protein cage, and the antigen is attached to said protein cage by covalent attachment, such as in a fusion of the antigen and the heat shock protein or by covalent attachment using a linker to connect the antigen to the heat shock protein. Examples of linkers include homo-bifunctional linkers, such as glutaraldehyde, dimethyl adipimidate (DMA), dimethyl suberimidate (DMS), dimethyl pimelimidate (DMP), N-hydroxysuccinimide (NHS), dithiobis(succinimidylpropionate (DSP), and dithiobis(sulfosuccinimidylpropionate) (DTSSP). Alternatively, hetero-bifunctional linkers, such as those with N-hydroxysuccinimide (NHS) at a first end and a free —SH at a second end. Examples of such hetero-bifunctional linkers include: [succinimidyl 3-(2-pyridyldithio)propionate](SPDP) or [succinimidyl trans-4-(maleimidylmethyl)cyclohexane-1-carboxylate](SMCC). Other types of linkers that may be used in the proactice of the invention include molecules that are polymers, peptides, carbohydrates, lipids, and nucleic acids. In some aspects, the linker is cleavable.

Examples of antigens used in the practice of this invention are bacterial antigens, mycobacterial antigens, viral antigens, and tumor antigens. Tumor antigens can include those expressed on melanoma cells, lymphoma cells, Hodgkin's Disease cells, anaplastic large cell cancer, prostate cancer cells, Burkitt's lymphoma cells, and cervical carcinoma cells. Examples of specific tumor antigens that may be used in the practice of this invention include those listed in Tables 1-7.

Alternatively, in some aspects, a viral antigen derived from viruses such as herpes simplex virus (HSV), hepatitis B virus (HBV), hepatitis C virus (HCV), cytomegalovirus (CMV), Epstein-Barr virus (EBV), influenza virus, measles virus, human immunodeficiency virus (HIV), and human papilloma virus (HPV) may be used. In further aspects, the antigen is an allergy antigen such as ragweed, grass, tree pollen, animal dander, or molds.

In additional aspects, the therapeutic vaccine can further comprises an adjuvant. The adjuvant can form part of the interior or exterior of a protein cage or the protein cage composition can be formulated in an admixture comprising the adjuvant. An adjuvant can take the form of a protein, a lipid, a lipoprotein, or a nucleic acid, specific examples of which include: lipid A, muramyl di-peptide (MDP), CpG motifs, or polyI/polyC, endotoxin, and lipopolysaccharide (LPS).

In various aspects, the adjuvant is attached with a linker to the heat shock protein. Examples of linkers include homo-bifunctional linkers, such as glutaraldehyde, dimethyl adipimidate (DMA), dimethyl suberimidate (DMS), dimethyl pimelimidate (DMP), N-hydroxysuccinimide (NHS), dithiobis(succinimidylpropionate (DSP), and dithiobis(sulfosuccinimidylpropionate) (DTSSP). Alternatively, hetero-bifunctional linkers, such as those with N-hydroxysuccinimide (NHS) at a first end and a free —SH at a second end. Examples of such hetero-bifunctional linkers include: [succinimidyl 3-(2-pyridyldithio)propionate](SPDP) or [succinimidyl trans-4-(maleimidylmethyl)cyclohexane-1-carboxylate](SMCC). Other types of linkers that may be used in the proactice of the invention include molecules that are polymers, peptides, carbohydrates, lipids, and nucleic acids. In some aspects, the linker is cleavable.

In additional aspects, the therapeutic vaccine can further comprises a targeting moiety, such as a targeting moiety that binds a cell surface molecule on antigen presenting cells (APC's). An example of such a targeting moiety is an antibody that binds the Fc receptor, a clathrin coated pit protein, a chemokine receptor, or a cytokine receptor APC's.

In a further embodiment, the present invention provides a method of providing a therapeutic vaccine for the treatment of a disease in a subject by administering to the subject a therapeutically effective amount of a therapeutic vaccine comprising a heat shock protein assembled into a protein cage and an antigen, thus providing treatment of the subject. In some aspects, the therapeutic vaccine induces cellular immunity. Examples of diseases that may be treated with the invention include: bacterial or fungal infectious diseases, acute or chronic viral infections, allergies, and cancers. In one aspect, the antigen is a cancer antigen.

In particular aspects, the therapeutic vaccine can further comprises an adjuvant, either as part of the interior or exterior of a protein cage, or the protein cage composition can be formulated in an admixture comprising the adjuvant. An adjuvant can take the form of a protein, a lipid, a lipoprotein, or a nucleic acid, specific examples of which include: lipid A, muramyl di-peptide (MDP), CpG motifs, or polyI/polyC, endotoxin, and lipopolysaccharide (LPS).

In some aspects, the administration can be by subcutaneous, intraperitoneal, intravenous, intraarterial, transdermal, transcutaneous, intranasal, topical, entereal, intravaginal, sublingual, or rectal administration.

In additional aspects, the method can further comprise the step of administering immunotherapy.

### BRIEF DESCRIPTION OF THE DRAWINGS

FIG. 1 illustrates the structural components of the sHsp protein cages of the invention.

FIG. 2 illustrates the processing of a Hsp-antigen protein cage through an antigen presenting cell (APC) to the MHC I pathway resulting in display of antigenic peptides on the APC surface in the context of MHC I. A cognate CD8+ T cell's T cell receptor binds to the presented antigen MHC I complex on the APC and becomes activated.

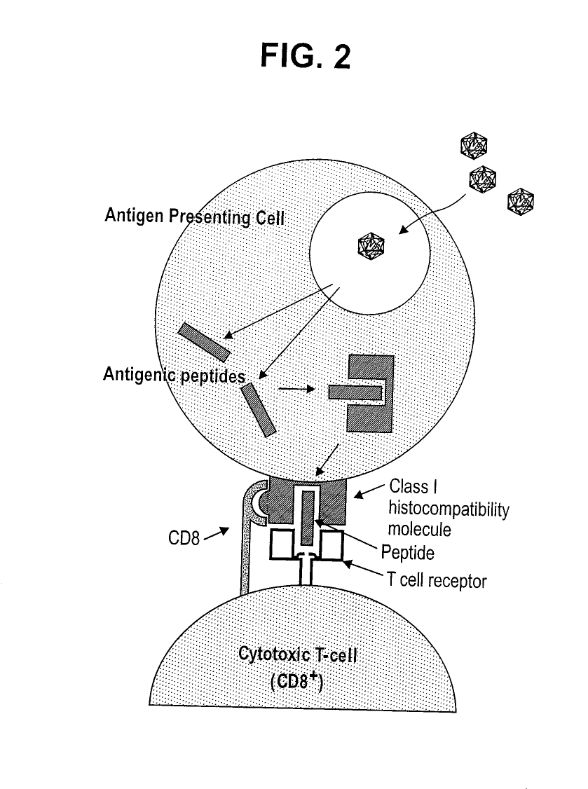

### DETAILED DESCRIPTION OF THE INVENTION

### I. Introduction

Heat shock proteins have features that allow their use in the development of prophylactic and therapeutic vaccines against a variety of conditions, such as cancers and infectious diseases. Among other activities, (1) heat shock proteins are capable of chaperoning peptides, including antigenic peptides; (2) interacting with specific receptors on antigen presenting cells (APC's); (3) stimulating APC's to secrete inflammatory cytokines; and (d) mediating maturation of dendritic cells. See, e.g., Srivastava, Vaccine, 19:2590-7 (2001); Singh et al., Biol. Chem., 382:629-36 (2001).

For example, Hsp's of molecular weights of about 60, 70, and 90 kDa have been shown to elicit a potent anticancer immune response mediated by the adaptive and innate immune system. The response of the immune system to Hsp's proceeds along two fronts. In one response, Hsp's stimulate the MHC I pathway of antigen presention in APC's. Thus, following receptor-mediated uptake of Hsp peptide complexes (e.g., Hsp70 and gp96) by APC's, peptides chaperoned by the Hsp are presented on the surface of APC's by MHC I molecules, which allows a CD8-specific T cell response to be induced. However, apart from their roles in chaperoning antigenic peptides to APC's, Hsp's by themselves appear to provide activating signals for APC's and natural killer (NK) cells. In particular, it has been shown that binding of peptide-free Hsp70 to Toll-like receptors on APC's results in the secretion of pro-inflammatory cytokines by APC's which results in the nonspecific stimulation of the immune system. Moreover, soluble, as well as, cell-membrane bound Hsp70 on tumor cells can directly activate the cytolytic and migratory capacity of NK cells. In addition to a role in cancer treatment, Hsp's have been shown to play a role in viral infections, including human and simian immunodeficiency virus (HIV and SIV), measles, and choriomeningitis. Additionally, Hsp's have been found to induce tolerance against autoimmune diseases. See, Multhoff, Handbook of Exp. Pharm., 172: 279-304 (2006).

Accordingly, a number of investigators have used heat shock proteins containing compositions for a variety of vaccine and immunotherapy applications. For example, various human papilloma viral (HPV) proteins have been fused to stress proteins such as Hsp65 and Hsp71 to treat the clinical manifestations of HPV infection. (See, e.g., U.S. Pat. Nos. 6,797,491; 6,900,035; 7,157,089.) Other heat shock proteins such as Hsp40 and Hsp70 have been used in combination with model antigens in vaccine preparations. (See, e.g., U.S. Pat. Nos. 6,641,812; 6,656,679; 6,663,868.) Mycobacterial Hsp-antigen fusions have also been described. (See, e.g., U.S. Pat. Nos. 6,335,183; 6,338,952.)

In an effort to improve the usefulness of Hsp-based vaccines for use in immunotherapy, the present inventors have found that the use of Hsp proteins for the formation of protein cages containing antigen provides an unexpectedly robust and highly specific immune response. Among the other features, the present inventors have used small heat shock proteins which are capable of undergoing assembly into oligomeric protein cages. Such Hsp protein cages afford a number of advantages, including the ability to shield labile antigens and/or adjuvants within the interior of protein cages. Furthermore, the subunits of such protein cages can be easily modified to include amino acids with reactive groups that allow the precise attachment of antigens and/or adjuvant to protein cages.

Protein cages have been described previously as protein “shells” that may be loaded with different types of materials. As an example, viral coat proteins have been used to form protein cages that encapsulate nonviral materials; see, U.S. Pat. Nos. 6,180,389 and 6,984,386, and U.S. Patent Application Publication No. 2004/0028694. Moreover, ferritin protein cages have been described that can be loaded with uniform materials. The adaptability of such proteins for this use results from their ability to form self-assembling shells. Additionally, many protein cages thus formed have natural controllable channels through which materials can pass, thus allowing for the reversible or irreversible loading of the protein cages. As discussed in greater detail herein, among the preferred proteins that may advantageously be used to form the protein cages of the present invention are Hsp's (in particular, the small Hsp's, “sHsp”) that are able to assemble into oligomers, allowing such compositions to serve as protein cages for the delivery of antigenic materials to the immune system, for the purposes of triggering cellular and humoral immunity.

### II. Components of Hsp Protein Cages for Immunotherapy

Accordingly, various embodiments of the present invention comprise a Hsp protein cage (a “ProteoCage”), an associated antigen, and optionally an adjuvant for stimulating the immune system to generate an immune response, e.g., the development of a cellular response to the associated antigen. As described in greater detail herein, the oligomeric structure of Hsp protein cages, having both an outside and inside aspect, allows flexibility in the configuration of antigens and adjuvants with respect to the structure of the protein cage. For example, antigens may be associated with the outside or inside or both of the protein cage. Similarly, adjuvants, when used, may also be associated with the outside or inside or both of the protein cage. The association of both antigens and adjuvants with protein cages can be noncovalent or covalent as described in greater detail herein. Furthermore, the adjuvant can be on the outside of the protein cages, while the antigen is on the inside, or vice versa. In another configuration, both the antigen and adjuvant are on the inside or outside of a protein cage, and in some embodiments both antigen and adjuvant are on both sides of the protein cage. The skilled artisan will appreciate that the unique three-dimensional topology of Hsp protein cages allows great flexibility in the placement of antigens and adjuvants in protein cages, depending on the needs of a given application. As an example, in the case of adjuvants that may be labile to hydrolysis under physiological conditions or by plasma or tissue enzymes, such as polyI/polyC or nucleic acids containing CpG motifs, it may be desirable to enclose such labile reagents within the interior of the Hsp protein cages. In such an embodiment, the antigen may also be enclosed within the interior of the protein cage or else, the antigen may be on the outside of the protein cage. In one advantageous embodiment, the antigen can be on the outside of the Hsp protein cage, while the adjuvant can be located on the inside.

Yet another advantage provided by the protein cages of the present invention for use in immunotherapy is the ability to target the protein cages to a distinct target cell (e.g., APC's). One feature of the Hsp protein cages of the present invention that allow this to occur is the fact that Hsp's have been shown to undergo receptor-mediated uptake into APC's, indicating the presence of specific Hsp receptors on cells (see, e.g., Hilf et al., Intl. J. Hyperthermia, 18:521-533 (2002)). Alternatively, a targeting moiety can be placed on the outside of an Hsp protein cage which can target protein cages to specific cell types, including APC's. Such targeting moieties are described in U.S. Provisional Patent Application No. 60/891,457, U.S. patent application Ser. No. 12/035,928, and PCT/US08/54,745, which are incorporated herein by reference in their entireties. As explained herein, such targeting moieties can be antibodies, ligands for receptors, lipids, or polysaccharides, among other chemical entities, that can direct the Hsp protein cages of the present invention to a cell type of interest. For example, an antibody that can target Hsp protein cages to a cell surface marker specific or highly expressed on the surface of APC's may be used to guide or home antigen and adjuvant containing protein cages to APC's. As alluded to above, the antigens and adjuvants of such protein cages can be in a variety of configurations, depending the particular application.

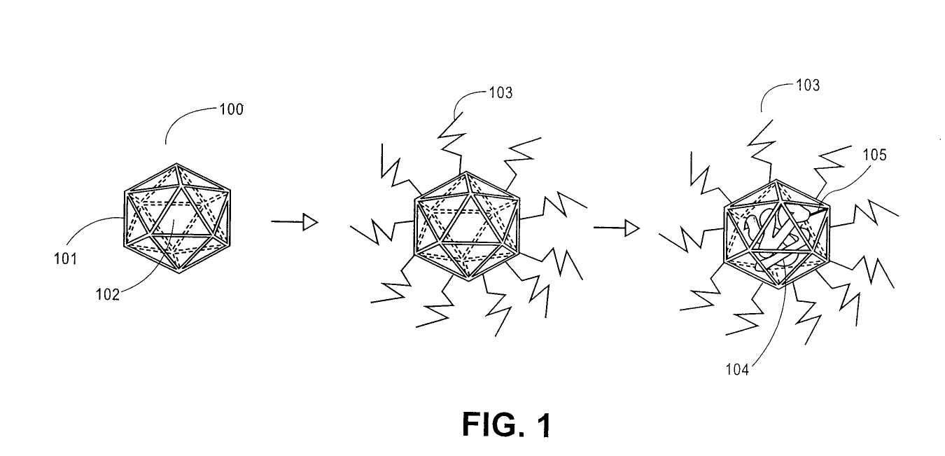

With reference to FIG. 1, a typical Hsp protein cage or ProteoCage 100 of the invention has an exterior 101 and interior aspect 102. An antigen molecule 104 can be encapsulated within the interior of the protein cage by covalent or noncovalent means. Structure 105 (segment in black) represents such a covalent linkage and can be either a chemical linker or a peptide sequence that is recombinantly fused between a Hsp subunit and an antigen. Alternatively, an antigen may be attached to the exterior of a protein cage, generally by covalently means, but also by recombinant means, such as via a linker or as a fusion protein with a heat shock protein subunit. Adjuvant, if present, may also be contained within the interior or attached to the exterior of a protein cage. Optionally, a targeting moiety 103 may be attached to the exterior of a protein cage, generally by covalent means. As discussed below, other optional reagents can be associated with protein cages, such as nucleic acid molecules that inhibit gene expression or that direct the expression of an exogenous protein, such as an antigen. It will be appreciated by the skilled artisan that the structures shown in FIG. 1 are not drawn to scale, and FIG. 1 is presented to illustrate the general structural features of the present invention.

We describe in greater detail below the components that may be used to form the Hsp protein cages of the present invention.

A. Heat Shock Proteins

In general, any heat shock protein capable of forming an oligomeric structure with an internal core space can be used in the practice of this invention. For example, in one embodiment, small heat shock proteins (sHsp's) may be used in the practice of this invention. sHsp's are ubiquitous among all organisms, with subunit sizes ranging from 12 to 42 kDa. sHsp's demonstrate amino acid sequence homology, with much of the conservation lying in a region known as the α-crystallin domain, which is a stretch of 80-100 amino acids generally located in the C-terminal part of the sequences of sHsp's (see, deJong et al., Int. J. Biol. Macromol., 22: 151-162 (1998)).

Among the known small heat shock proteins that may be used in the formation of protein cages is the small heat shock protein of Methanococcus jannaschii, which assembles into a 24 subunit cage with 432 symmetry (see, Kim et al., Nature, 394:595-599 (1998); Kim et al., J. Struct. Biol., 121:76-80 (1998); and Kim et al., PNAS, 95:9129-9133 (1998)). Protein cages formed from the Hsp16.5 protein of M. Jannaschii have a 12 nm exterior diameter and a 6.5 nm interior diameter. Such protein cages are stable to heat (up to 65° C.) and pH in the range of 6-9. Gel filtration chromatography indicates that Hsp16.5 is monodisperse, and a symmetry test on Hsp 16.5 indicated that the protein cage formed from this Hsp had at least a loose octahedral symmetry (see, Haley et al., J. Mol. Biol., 298:261-72 (2000)).

Similarly, human Hsp27 can also be used to form the protein cages of the invention. In contrast to the structures formed from the Hsp16.5 protein of M. Jannaschii, human Hsp27 displays more polydisperse characteristics on gel filtration, with a wide range of diameters, and a weaker octahedral symmetry (see, Haley et al., J. Mol. Biol., 298:261-72 (2000)).

The small heat shock proteins of Mycobacterium tuberculosis can also be used in the practice of this invention. Mycobacterium tuberculosis has two small heat shock proteins, Acr1 (alpha-crystallin-related protein 1, or Hsp16.3/16-kDa antigen) and Acr2 (HrpA), both of which are highly expressed under different stress conditions. Nanoelectrospray mass spectrometry showed that Acr2 formed a range of oligomers composed of dimers and tetramers, whereas Acr1 was a dodecamer. Electron microscopy of Acr2 showed a variety of particle sizes. Using three-dimensional analysis of negative stain electron microscope images, it has been demonstrated that Acr1 forms a tetrahedral assembly with 12 polypeptide chains. See, Kennaway et al., J. Biol. Chem., 280:33419-25 (2005).

A small heat shock protein from a hyperthermophilic archaeum, Thermococcus sp. strain KS-1 can also be used in the practice of this invention. Electron microscopy revealed that the protein exists as a spherical oligomer with a diameter of 14±1 nm. The molecular weight of the oligomer was determined to be 478.6 kDa by size exclusion chromatography-multiangle laser light scattering. Thus, the Thermococcus sHsp is likely to exist as a spherical 24meric oligomer with almost the same structure as the Methanococcus jannaschii sHsp as described above. See, Usui et al., J. Biosci. and Bioeng., 92: 161-166 (2001).

Two sHsp's (StHsp19.7 and StHsp14.0) from a thermoacidophilic crenarchaeon, Sulfolobus tokodaii strain 7 can also be used in the practice of this invention. StHsp19.7 forms a filamentous structure consisting of spherical particles and lacks molecular chaperone activity. Fractionation of Sulfolobus extracts by size exclusion chromatography with immunoblotting indicates that StHsp19.7 exists as a filamentous structure in vivo. In contrast, StHsp14.0 exists as a spherical oligomer like other sHsp's. StHsp14.0 forms variable-sized complexes an enzyme at 90° C. See, Usui et al., Protein Science, 13:134-144 (2004).

A sHsp has been purified from yeast which has characteristics favorable for the practice of this invention. A 26 kDa protein was purified to apparent homogeneity from Saccharomyces cerevisiae with a recovery of 74% using a three step procedure consisting of ethanol precipitation, sucrose gradient ultracentrifugation, and heat inactivation of residual contaminants. Analysis of the purified protein by electron microscopy revealed near spherical particles with a diameter of 12.0 nm (n=57, standard deviation +/−1.6 nm), displaying a dispersion in size ranging from 9.2 to 16.1 nm, identical to Methanococcus jannaschii Hsp16.5. See, Ferreira et al., Protein Expr. Purif., 47:384-92 (2006).

Another yeast sHsp that can be used in the practice of this invention is SpHsp16.0 from Schizosaccharomyces pombe. Analysis of the purified protein revealed it to be a hexadecameric globular oligomer near the physiological growth temperature.

Hsp16.9 from wheat can also be used in the practice of the invention. The X-ray crystal structure of Hsp16.9 has been determined and shows that Hsp16.9 exists as a dodecamer. The dodecamer consists of two disks, each comprising six α-crystalline domains organized as a trimer of dimers. A comparison of the structure of wheat Hsp 16.9 and M. jannaschii Hsp 16.5 is provided in van Montfort et al., Nature Structural Biology, 8:1025-1030 (2001).

While a number of heat shock proteins have been specifically discussed above in the specification for exemplary purposes, the skilled artisan will recognize that this list is merely a nonlimiting subset of heat shock proteins that may be used in the practice of this invention. Indeed, any heat shock protein capable of forming an oligomeric structure with an internal core space can be used in the practice of this invention. Further examples of Hsp's that may be used in the practice of this invention include, without limitation, those from a variety of organisms meeting this definition as listed below.

A nonlimiting list of suitable small Hsp's is provided herewith the species of organism indicated followed by the number of homologues and accession numbers indicated in parentheses: M. thermautotrophicus (1) (AAB85357); M. acetivorans STR C2A (3) (NP_{—}618465, NP_{—}615107, NP_{—}619401); M. mazei Goel (4) (NP_{—}633443, NP_{—}632985, NP_{—}632984, NP_{—}632507); M. jannaschii (1) Q57733; M. kandleri AV19 (1) AAM01219; S. solfataricus (2) (NP_{—}343781, NP_{—}343935); S. tokodaii (2) (NP_{—}376442, NP_{—}377625); A. pernix (2) (APE1950, APE0103); P. aerophilum (3) (NP_{—}560543, NP_{—}560503, NP_{—}559894); T. acidophilum (2) (CAC11613, CAC11993); T. volcanium (3) (NP_{—}111503, NP_{—}393915, NP_{—}111294); A. fulgidus (2) (G69411, B69496); P. abyssi (1) (NP_{—}126108); P. furiosus (1) (NP_{—}579612); P. horikoshii (1) (D71196); Thermococcus KS-1 (1) (BAB40930) Halobacterium NRC-1 (5) (AAG20020, AAG18726, AAG20865, AAG19995, AAG18869); A. aeolicus (1) (A70411); T. elongates (1) (NP_{—}681663); T. tengcongensis (1) (NP_{—}624099); S. enterica (1) (NP_{—}456260); O. sativa (1) (CAA43210); T. aestivum (1) (CAA45902); H. sapiens α-crystallins (2) (NP_{—}000385, NP_{—}001876). (See, Laksanalamai et al., Extremophiles, 2004, 8: 1-11.)

Additional small Hsp's for use in the practice of this invention are provided as follows as a listing of species and accession numbers or journal references: Homo sapiens α A-crystallin (PO₂₄₈₉), H. sapiens α B-crystallin (P02511), H. sapiens Hsp20 (Kato et al. J. Biol. Chem., 269: 15302-9 (1994)), Homo sapiens Hsp27 (P04792), P. lucida Hsp27 (U85501), Drosophila melanogaster Hsp27 (P02518), D. melanogaster 1(2)efl (X77635), Artemia franciscana p26 (Liang et al., J. Biol. Chem., 272: 19051-8 (1997)), Xenopus laevis Hsp30c (P30218), Poeciliopsis lucida Hsp30b (U85502), Halocynthia roretzi HR-29 (JX0258), Schistosoma mansoni p40-2 N- and C-terminal domains (M96866), H. sapiens Hsp127 (U15590), Caenorhabditis elegans Hsp16 (P06581), C. elegans Hsp12.3 (Z68342), Acanthocheilonema viteae AV25 (S29691), Saccharomyces cerevisiae Hsp26 (M23871), S. cerevisiae Hsp42 (U41401), Neurospora crassa Hsp30 (M55672), Pisum sativum chloroplast Hsp21 (P09886), P. sativum mitochondrial Hsp22 (P46254), P. sativum cytoplasmic class I Hsp18 (P19243), P. sativum endoplasmic reticulum Hsp22 (P19244), P. sativum cytoplasmic class II Hsp17 (P19242), Chlamydomonas reinhardtii Hsp22 (×15053), Toxoplasma gondii Hsp30:BAG1 (Z48750), Bradyrhizobium japonicum HspA (U55047), Escherichia coli IbpA (A45245), Buchnera aphidicola Ibp (Y11966), Legionella pneumophila GspA (S49042), Leuconostoc oenos Lo18 (Jobin et al., Appl. Environ. Microbiol., 63: 609-14 (1997)), Clostridium acetobutylicum Hsp18 (S25534), Methanococcus jannaschii Hsp20 (U67483), Bradyrhizobium japonicum HspC (U55047), Stigmatella aurantiaca SP21 (M94510), Streptomyces albus Hsp18 (U17419), Mycobacterium leprae 18K (M22587), M. tuberculosis 14K (A42651), Bacillus subtilis CotM (U72073). See, deJong et al., Int. J. Biol. Macromol., 22: 151-162 (1998).

B. Antigens

In general, any preparation of crude or purified material that contains a material capable of generating an immune response can be used in the practice of this invention. It is well known in the art that an immune response can be raised against any number of cellular components, such as proteins, peptides, carbohydrates, lipids, and nucleic acids. It will be appreciated that preparations of antigens can include crude cell or viral lysates or partially or substantially purified components thereof. Thus, for instance, for the purposes of cancer immunotherapy, a whole cell or tissue lysate, or a membrane extract isolated therefrom may be desired. Examples of cancers that may be used in the practice of this invention include, without limitation: carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CML), multiple myeloma, mantle cell lymphoma, Waldenstrom's macrogobulinemia, and Philadelphia positive cancers.

Alternatively, specific purified antigens can be used. Examples of cancer cell antigens include, without limitation, those derived from the cancers listed above as well as: melanoma, lymphoma, Hodgkin's Disease, anaplastic large cell cancer, prostate cancer, Burkitt's lymphoma, and cervical carcinoma.

Examples of specific cancer cell antigens include, without limitation, those disclosed in Tables 1-7 below (see, e.g., Novellino et al., Cancer Immunol. Immunother., 54: 187-207 (2005)). The reference numbers in the table below refer to the references cited in Novellino et al.

TABLE 1

Class I HLA-restricted cancer-testis antigens. These
antigens were found to be expressed by normal spermatocytes
and/or spermatogonia of testis. Occasionally, MAGE-3,
MAGE-4 and the GAGE genes were found to be expressed also
in placenta [38, 40]. The NY-ESO-1 antigen was found to be
expressed also in normal ovary cells [30]

| | HLA | Peptide | | Tissue distribution |
|---|---|---|---|---|
| Gene | allele | epitope | References | among tumors^{a} |
| BAGE | Cw16 | AARAVFLAL | Boël et al. [16] | Melanoma, myeloma (stage |
| | | | | III); lung, bladder, and breast |
| | | | | carcinomas; H/N SCC,^{b} |
| | | | | NSCLC^{b} |
| CAMEL | A2 | MLMAQEALAFL | Aarnoudse et al. [1] | Melanoma, myeloma (stage |
| | | | | III); NSCLC, H/N SCC, |
| | | | | esophageal SCC, infiltrating |
| | | | | bladder carcinoma, prostate |
| | | | | and breast carcinoma; sarcoma |
| DAM-6, -10 | A2 | FLWGPRAYA | Fleischhauer et al. [52] | Melanoma, skin tumors, |
| (MAGE- | | | | mammary and ovarian |
| B1, B2) | | | | carcinomas [115]; lung |
| | | | | carcinoma [39, 115]; |
| | | | | seminomas [39] |
| GAGE-1, -2, | Cw6 | YRPRPRRY | Van den Eynde et al. | Melanoma; myeloma (stage |
| -8 | | | [186]and De Backer et | III), lung carcinoma, H/N |
| | | | al. [40] | SCC, esophageal SCC; |
| | | | | infiltrating bladder carcinoma, |
| | | | | prostate^{b} and breast^{b} |
| | | | | carcinomas; sarcoma^{b} |
| GAGE-3, -4, | A29 | YYWPRLPRRY | De Backer et al. [40] | Similar to GAGE-1, -2, -8 |
| -5, -6, -7B | | | | |
| IL-13Rα2 | A*0201 | WLPFGFILI | Okano et al. [133] | Glioblastoma multiforme |
| MAGE-A1 | A1 | EADPTGHSY | Traversari et al. [181] | Melanoma, myeloma (stage |
| | A3 | SLFRAVITK | Chaux et al. [28] | III), lung carcinoma, H/N |
| | A24 | NYKHCFPEI | Fujie et al. [53] | SCC, esophageal SCC, |
| | A28 | EVYDGREHSA | Chaux et al. [28] | superficial and infiltrating |
| | B37 | REPVTKAEML | Tanzarella et al. [171] | bladder carcinoma; prostate,^{b} |
| | B53 | DPARYEFLW | Chaux et al. [28] | colorectal,^{b} and breast^{b} |
| | Cw2 | SAFPTTINF | Chaux et al. [28] | carcinomas, sarcoma.^{b} (For |
| | Cw3 | SAYGEPRKL^{c} | Chaux et al. [28] | minor pattern of expressions, |
| | Cw16 | SAYGEPRKL^{c} | van der Bruggen et al. | also see [41, 42, 188]) |
| | | | [190] | |
| MAGE-A2 | A2 | KMVELVHFL | Visseren et al. [193] | The same as MAGE-A1 |
| | A2 | YLQLVFGIEV | Visseren et al. [193] | |
| | A24 | EYLQLVFGI | Tahara et al. [168] | |
| | B37 | REPVTKAEML | Tanzarella et al. [171] | |
| MAGE-A3 | A1 | EADPIGHLY | Gaugler et al. [56] | The same as MAGE-A1 |
| | A2 | FLWGPRALV | van der Bruggen et al. | |
| | | | [189] | |
| | A24 | TFPDLESEF | Oiso et al. [131] | |
| | A24 | IMPKAGLLI | Tanaka et al. [169] | |
| | B44 | MEVDPIGHLY | Herman et al. [68] and | |
| | | | Fleischhauer et al. [51] | |
| | B52 | WQYFFPVIF | Russo et al. [154] | |
| | 837 | REPVTKAEML | Tanzarella et al. [171] | |
| | B*3501 | EVDPIGHLY | Benlalam et al. [14] | |
| MAGE-A4 | A2 | GVYDGREHTV | Duffour et al. [48] | The same as MAGE-A1 |
| MAGE-A6 | A34 | MVKISGGPR | Zorn and Hercend [220] | The same as MAGE-A1 |
| | B37 | REPVTKAEML | Tanzarella et al. [171] | |
| | B*3501 | EVDPIGHVY | Benlalam et al. [14] | |
| MAGE-A10 | A2 | GLYDGMEHL | Huang et al. [73] | The same as MAGE-A1, with |
| | | | | the exception of colorectal and |
| | | | | breast carcinomas |
| MAGE-A12 | Cw7 | VRIGHLYIL | Panelli et al. [136] and | The same as MAGE-A1 |
| | | | Heidecker et al. [67] | |
| NA88-A | B13 | MTQGQHFLQKV | Moreau-Aubry et al. | Melanoma |
| | | | [120] | |
| NY-ESO-1 | A2 | SLLMWITQCFL | Jäger et al. [77] | The same as CAMEL |
| | A2 | SLLMWITQC | Jäger et al. [77] | |
| | A2 | QLSLLMWIT | Jäger et al. [77] | |
| | B*3501 | MPFATPMEA | Benlalam et al. [14] | |
| NY-ESO-1a | A31 | ASGPGGGAPR | Wang et al. [204] | |
| (CAG-3) | | | | |
| SSX-2 | A2 | KASEKIFYV | Ayyoub et al. [8] | Melanomas; lymphomas; H/N, |
| | | | | colon carcinomas |
| TRAG-3 | A*0201 | ILLRDAGLV | Zhu et al. [218] | Melanomas; leukemias; |
| | | | | NSCLC, prostate and breast |
| | | | | carcinomas |

^{a}See also van der Bruggen et al. [191] for a more detailed tissue distribution
^{b}These epitopes share different HLAs-that is they are recognized by specific T cells when presented by different HLA alleles. This phenomenon is important, as it allows an epitope to be employed for cancer immunotherapy in a larger number of patients
^{c}Frequency of expression less than 10%

TABLE 2

Class I HLA-restricted differentiation antigens. These TAAs
can only be expressed in normal and neoplastic cells of the
same lineage. Those antigens which also present class II
HLA-restricted epitopes are in bold type

| | HLA | Peptide | | Normal |
|---|---|---|---|---|
| Gene | allele | epitope | References | tissue/tumors |
| CEA | A2 | YLSGANLNL | Tsang et al. [183] | Embryonic tissue; normal |
| | | (CAP-1)^{a} | | epithelia differentiation |
| | A3 | HLFGYSWYK | Kawashima et al. [92] | overexpressed in colon and |
| | | | | other adenocarcinomas |
| Ep-CAM | A2 | GLKAGVIAV | Nagorsen et al. [123] | Epithelia overexpressed in |
| | | | | colon and other |
| | | | | adenocarcinomas |
| Gp100 | A2 | KTWGQYWQV | Bakker et al. [11] | Melanocyte/melanoma |
| | A2 | AMLGTHTMEV | Tsai et al. [182] | |
| | A2 | MLGTHTMEV | Tsai et al. [182] | |
| | A2 | SLADTNSLAV | Tsai et al. [182] | |
| | A2 | ITDQVPFSV | Kawakami et al. [86] | |
| | A2 | LLDGTATLRL | Kawakami et al. [85] | |
| | A2 | YLEPGPVTA | Cox et al. [38] | |
| | A2 | VLYRYGSFSV | Kawakami et al. [86] | |
| | A2 | RLMKQDFSV | Kawakami et al. [88] | |
| | A2 | RLPRIFCSC | Kawakami et al. [88] | |
| | A3 | LIYRRRLMK | Kawakami et al. [88] | |
| | A3 | ALNFPGSQK | Kawashima et al. [91] | |
| | A3 | SLIYRRRLMK | Kawashima et al. [91] | |
| | A3 | ALLAVGATK | Skipper et al. [165] | |
| | A24 | VYFFLPDHL | Robbins et al. [149] | |
| | A*6801 | HTMEVTVYHR | Sensi et al. [163] | |
| | B*3501 | VPLDCVLYRY | Benlalam et al. [14] | |
| | Cw8 | SNDGPTLI | Castelli et al. [27] | |
| Mammaglobin- | A3 | PLLENVISK | Jaramillo et al. [79] | Mammary gland/breast |
| A | | KLLMVLMLA | Jaramillo et al. [79] | cancer |
| | | TTNAIDELK | Jaramillo et al. [79] | |
| | | AIDELKECF | Jaramillo et al. [79] | |
| Melan-A/ | A2 | AAGIGILTV | Coulie et al. [36] and | Melanocyte/melanoma |
| MART-1^{b} | | | Kawakami et al. [83] | |
| | A2 | EAAGIGILTV | Schneider et al. [162] | |
| | A2 | ILTVILGVL | Castelli et al. [26] | |
| | B*3501 | | Benlalam et al. [14] | |
| | B45 | AEEAAGIGIL | Schneider et al. [162] | |
| | B45 | AEEAAGIGILT | Schneider et al. [162] | |
| MC1R | A2 | TILLGIFFL | Salazar-Onfray et al. [156] | Melanocyte/melanoma |
| | A2 | FLALIICNA | Salazar-Onfray et al. [156] | |
| OA1 | A*2402 | LYSACFWWL | Touloukian et al. [180] | Melanocyte/melanoma |
| P polypeptide | A2 | IMLCLIAAV | Touloukian et al. [179] | Melanocyte/melanoma |
| PSA | A1 | VSHSFPHPLY | Corman et al. [34] | Prostate gland/prostate |
| | A2 | FLTPKLKLQCV | Correale et al. [35] | carcinoma |
| | A2 | VISNDVCAQV | Correale et al. [35] | |
| TRP-1 (or | A31 | MSLQRQFLR | Wang et al. [202] | Melanocyte/melanoma |
| gp75) | | | | |
| TRP-2 | A2 | SVYDFFVWL^{c} | Parkhurst et al. [137] | Melanocyte/melanoma |
| | A2 | TLDSQVMSL | Noppen et al. [125] | |
| | A31 | LLGPGRPYR^{d} | Wang et al. [201] | |
| | A33 | LLGPGRPYR^{d} | Wang et al. [203] | |
| | Cw8 | ANDPIFVVL | Castelli et al. [27] | |
| Tyrosinase | A1 | KCDICTDEY | Kittlesen et al. [99] | Melanocyte/melanoma |
| | A1 | SSDYVIPIGTY | Kawakami et al. [88] | |
| | A2 | YMDGTMSQV | Wolfel et al. [208] | |
| | A2 | MLLAVLYCL | Wolfel et al. [208] | |
| | A24 | AFLPWHRLF | Kang et al. [81] | |
| | B44 | SEIWRDIDF | Brichard et al. [20] | |
| | B*3501 | TPRLPSSADVEF | Benlalam et al. [14] | |

^{a}CAP-1 is an alternative name of this peptide
^{b}Two different groups simultaneously discovered this gene and gave it two different names: MART-1 [84]and Melan-A [36], respectively
^{c}This peptide was shown to be a CTL target also in glioblastoma multiforme restricted by HLA-A2 [111]
^{d}These epitopes share different HLA-A3 subtypes. This allows an epitope to be employed for cancer immunotherapy in a larger number of patients

TABLE 3

Class I HLA-restricted, widely occurring, overexpressed
TAAs. Underlined amino acids in the epitopes indicate
splicing aberration. Those antigens which also present
class II HLA-restricted epitopes are in bold type

| | HLA | Peptide | | Tissue distribution | |
|---|---|---|---|---|---|
| Gene | allele | epitope | References | Tumors | Normal tissues |

| | | | | | |
|---|---|---|---|---|---|
| Adipo- | A2 | SVASTITGV | Schmidt et al. [159] | RCC, melanoma; | Adipocytes, |
| philin | | | | breast, colon, and | macrophages |
| | | | | ovarian carcinomas; | |
| | | | | CML, multiple | |
| | | | | myeloma | |
| AIM-2^{a} | A1 | RSDSGQQARY | Harada et al. [66] | Melanoma; | Weakly expressed in |
| | | | | neuroblastoma; Ewing's | lung, brain, liver, and |
| | | | | sarcoma; breast, | testis |
| | | | | ovarian, and colon | |
| | | | | carcinomas | |
| AFP | A2 | GVALQTMKQ | Butterfield et al. [22] | Hepatocellular | Synthesized by the |
| | | | | carcinoma, and yolk- | fetal liver and yolk |
| | | | | sac tumors. Also | sac. Low levels in |
| | | | | detected in hilar bile | adult brain, heart, |
| | | | | duct carcinoma; | skeletal muscle, |
| | | | | pleomorphic adenoma | prostate, stomach, |
| | | | | of parotid gland; | pancreas, adrenal |
| | | | | prostate, pancreatic, | gland, salivary gland, |
| | | | | bladder, and thyroid | liver, small intestine, |
| | | | | papillary carcinomas | and peripheral blood |
| | | | | [75] | [75] |
| ART-4 | A24 | AFLRHAAL | Kawano et al. [90] | Lung, esophageal, H/N, | High expression in |
| | | DYPSLSATDI | Kawano et al. [90] | gastric, cervical, | fetal liver, adult |
| | | | | endometrial, ovarian, | pancreas, and ovary. |
| | | | | and breast cancers; | Significant expression |
| | | | | leukemias | in heart, brain, |
| | | | | | placenta, liver, lung, |
| | | | | | kidney, spleen, |
| | | | | | thymus, prostate, |
| | | | | | testis, small intestine, |
| | | | | | colon, and PBMCs |
| CLCA2 | A2 | LLGNCLPTV | Konopitzky et al. | SCLC; pancreatic, and | Lung (very low levels |
| | | | [105] | esophageal carcinomas | by Northem blot), |
| | | SLQALKVTV | Konopitzky et al. | | trachea, mammary |
| | | | [105] | | gland |
| Cyp-B | A24 | KFHRVIKDF | Gomi et al. [60] | NSGLC; T-cell | Ubiquitously |
| | | DFMIQGGDF | Gomi et al. [60] | leukemia; | expressed in normal |
| | | | | lymphosarcoma; | tissues |
| | | | | bladder, ovarian, | |
| | | | | uterine, and esophageal | |
| | | | | carcinomas | |
| EphA2 | A*0201 | IMNDMPIYM | Alves et al. [2] | Overexpressed in | Lung, kidney, skin, |
| | | VLAGVGFFI | | breast, colon, lung, | ovary, thymus |
| | | | | prostate, and gastric | |
| | | | | carcinomas; metastatic | |
| | | | | melanomas; tumor | |
| | | | | neovasculature | |
| FGF-5 | A3 | NTYASPRFK^{b} | Hanada et al. [65] | RCC; prostate, and | Brain and kidney (low |
| | | | | breast carcinomas | expression) |
| G250 | A2 | HLSTAFARV | Vissers et al. [194] | RCC; colon, ovarian, | Epithelial cells of |
| | | | | and cervical carcinomas | gastric mucosa |
| GnT-V | A2 | VLPDVFIRC(V)^{c} | Guilloux et al. [63] | Melanoma; brain | Breast and brain (low |
| | | | | tumors; sarcoma | expression) |
| HER-2/neu | A2 | KIFGSLAFL | Fisk et al. [50] | Melanoma, ovarian, | Epithelial cells |
| | A2 | IISAVVGIL | Peoples et al. [142] | gastric, pancreatic | |
| | A2 | RLLQETELV | Kono et al. [104] | [14l],^{d} and breast | |
| | A2 | VVLGVVFGI | Rongcun et al. [151] | carcinomas | |
| | | ILHNGAYSL | Rongcun et al. [151] | | |
| | | YMIMVKCWMI | Rongcun et al. [151] | | |
| | A24 | TYLPTNASL | Okugawa et al. [134] | | |
| | A3 | VLRENTSPK | Kawashima et al. | | |
| | | | [92] | | |
| HST-2 | A31 | YSWMDISCWI | Suzuki et al. [167] | Gastric signet-ring cell | Not determined |
| (FGF-6) | | | | carcinoma | |
| hTERT | A2 | ILAKFLHWL | Vonderheide et al. | Lung, prostate, and | Hematopoietic stem |
| | | | [195] | ovarian carcinomas; | cells and progenitors; |
| | A2 | ILAKFLHWL | Minev et al. [119] | multiple myeloma; | germinal center cells; |
| | | RLVDDFLLV | Minev et al. [119] | melanoma; sarcoma; | basal keratinocytes; |
| | A3 | KLFGVLRLK | Vonderheide et al. | acute leukemias; non- | gonadal cells; certain |
| | | | [196] | Hodgkin's lymphomas | proliferating epithelial |
| | | | | | cells |
| iCE | B7 | SPRWWPTCL | Ronsin et al. [152] | RCC | Kidney, colon, small |
| | | | | | intestine, liver, heart, |
| | | | | | pituitary gland, |
| | | | | | adrenal gland, |
| | | | | | prostate, stomach |
| Livin | A2 | SLGSPVLGL | Schmollinger et al. | High levels in | Two isoforms. |
| (ML-IAP) | | RLASFYDWPL | [161] | melanoma [7, 197], | Expressed during |
| | | | | colon, and prostate | normal fetal |
| | | | | carcinomas, B-cell | development. |
| | | | | lymphomas, | Detected in adult |
| | | | | erythroleukemia and | heart, testis, ovary, |
| | | | | promyelocytic | thymus, spleen, |
| | | | | leukemia. Lower | lymph node, PBLs, |
| | | | | expression in breast and | and bone marrows. |
| | | | | cervical carcinomas, | Low levels in |
| | | | | and AML [7]. Good | prostate, small |
| | | | | expression in | intestine, colon, brain, |
| | | | | superficial bladder | placenta, liver, |
| | | | | cancer (and not in | skeletal muscle, |
| | | | | normal tissue) [58] | kidney, and pancreas. |
| | | | | | Not detectable in |
| | | | | | other adult tissues, |
| | | | | | including |
| | | | | | melanocytes [197]. A |
| | | | | | different pattern of |
| | | | | | expression is given by |
| | | | | | other authors by |
| | | | | | means of RT-PCR |
| | | | | | analyses: fetal |
| | | | | | kidney, heart, and |
| | | | | | spleen. In adult |
| | | | | | tissues: high levels in |
| | | | | | heart, placenta, lung, |
| | | | | | spleen, and ovary. |
| | | | | | Low levels in brain, |
| | | | | | skeletal muscle, |
| | | | | | kidney, and PBLs [7] |
| M-CSF | B*3501 | LPAVVGLSPGE | Probst-Kepper et al. | RCC | Liver, kidney |
| | | QEY^{e} | [145] | | |
| MUC1 | A11 | STAPPAHGV | Domenech et al. | Aberrantly glycosylated | Ductal epithelial cells |
| | | | 1995 [45] | forms in breast or | and activated T cells |
| | A2 | STAPPVHNV | Brossart et al. [21] | ovarian cancer | |
| MUC2 | A2 | LLNQLQVNL | Bohm et al. [17] | Ovary, pancreas, and | Colon, small |
| | | MLWGWREHV | Bohm et al. [17] | breast mucinous | intestine, bronchus, |
| | | | | tumors; colon | cervix, and gall |
| | | | | carcinoma of bladder | |
| | | | | nonmucinous type | |
| PRAME | A24 | LYVDSLFFL | Ikeda et al. [74] | Melanoma; H/N and | Testis, endometrium, |
| | A2 | VLDGLDVLL | Kessler et al. [93] | lung SCC; NSCLC | ovary, adrenals, |
| | | SLYSFPEPEA | Kessler et al. [93] | [185]; RCC; sarcoma; | kidney, brain, and |
| | | ALYVDSLFFL | Kessler et al. [93] | leukemias [184] | skin |
| | | SLLQHLIGL | Kessler et al. [93] | | |
| PSM A | A1 | HSTNGVTRIY | Corman et al. [34] | Prostate cancer; tumor- | Prostate epithelium |
| | A24 | LYSDPADYF | Horiguchi et al. [72] | associated | (cytosolic and PSMA- |
| | | NYARTEDFF | Horiguchi et al. [72] | neovasculature of | 2 isoform), ventral |
| | | | | several solid tumors | striatum and brain |
| | | | | | stem (PSMA-2 |
| | | | | | isoform), liver |
| | | | | | (PSMA-2 isoform), |
| | | | | | small intestine, |
| | | | | | kidney, spleen, and |
| | | | | | colon |
| P15 | A24 | AYGLDFYIL | Robbins et al. [147] | Melanoma | Testis, spleen, |
| | | | | | thymus, liver, kidney, |
| | | | | | lung, and retina |
| P53 | A24 | AIYKQSQHM | Umano et al. [184] | Esophageal, gastric, | Ubiquitous (low |
| | B46 | SQKTYQGSY^{f} | Azuma K et al. [10] | colon, pancreatic, and | level) |
| | | | | gall bladder carcinomas | |
| RAGE | B7 | SPSSNRIRNT | Gaugler et al. [57] | Melanoma; sarcomas; | Retina only |
| | | | | mesotheliomas; H/N | |
| | | | | tumors; bladder, renal, | |
| | | | | colon, and mammary | |
| | | | | carcinomas | |
| RUI | B51 | VPYGSFKHV | Morel et al. [121] | Melanoma; renal and | Testis, kidney, heart, |
| | | | | bladder carcinomas | skin, brain, ovary, |
| | | | | | liver, lung, |
| | | | | | lymphocytes, thymus, |
| | | | | | fibroblasts |
| RU2 | B7 | LPRWPPPQL | Van den Eynde et al. | Melanoma; sarcomas; | Testis, kidney, liver, |
| | | | [187] | leukemia; brain, | and urinary bladder |
| | | | | esophageal and H/N | |
| | | | | tumors; renal, colon, | |
| | | | | thyroid, mammary, | |
| | | | | bladder, prostatic, and | |
| | | | | lung carcinomas | |
| SART-1 | A24 | EYRGFTQDF | Kikuchi et al. [97] | H/N SCC; esophageal | Proliferating cells |
| | A*2601 | KGSGKMKTE | Shichijo et al. [164] | SCC; NSCLC; uterine | during the M phase. |
| | | | | cancer | Fetal liver; adult |
| | | | | | testis, heart, placenta, |
| | | | | | skeletal muscle, |
| | | | | | pancreas, spleen, |
| | | | | | thymus, prostate, |
| | | | | | uterus, and small |
| | | | | | intestine [164] |
| SART-2 | A24 | DYSARWNEI | Nakao et al. [124] | H/N SCC; esophageal | Although no |
| | | AYDFLYNYL | Nakao et al. [124] | SCG; lung | significant expression |
| | | SYTRLFLIL | Nakao et al. [124] | adenocarcinoma; | was observed at |
| | | | | melanoma; RCC; | protein level by |
| | | | | uterine | Western blot in |
| | | | | adenocarcinoma; brain | different tissues, high |
| | | | | tumors | mRNA expression |
| | | | | | was observed by |
| | | | | | Northern blot in heart, |
| | | | | | placenta, spleen, and |
| | | | | | ovary. Whereas a |
| | | | | | lower mRNA |
| | | | | | expression was seen |
| | | | | | in lung, skeletal |
| | | | | | muscle, kidney, testis, |
| | | | | | small intestine, and |
| | | | | | PBLs |
| SART-3 | A24 | VYDYNCHVDL | Yang et al. [211j | The same as SART-2 | The same as SART-2 |
| | | AYIDFEMKI | Yang et al. [211] | | |
| | A2 | LLQAEAPRL | Ito et al. [76] | | |
| | | RLAEYQAYI | Ito et al. [76] | | |
| SOXI0 | A2 | SAWISKPPGV | Khong and | Overexpressed in | Abundantly expressed |
| | | | Rosenberg, 2002 [95] | melanomas | in migratory neural |
| | | | | | crest during early |
| | | | | | stages of |
| | | | | | development. In |
| | | | | | adult, expression |
| | | | | | found in melanocytes, |
| | | | | | brain, heart, lungs, |
| | | | | | adrenal and salivary |
| | | | | | glands, colon, |
| | | | | | intestine, bladder, |
| | | | | | pancreas, prostate, |
| | | | | | and testis |
| Survivin | A2 | ELTLGEFLKL | Andersen et al. [3], | Abundantly expressed | Expressed during |
| | | | Schmitz et al. [1601, | in carcinomas (NSCLC | normal fetal |
| | | | Andersen et al. [4], | and SSC of the lung; | development. High |
| | | | Casati et al. [25], and | esophagus, liver, | expression in testis, |
| | | | Schmidt et al. [158] | pancreas, colon, breast, | thymus, and placenta. |
| | A2 | TLPPAWQPFL | Schmitz et al. [160] | ovary, bladder, and | Low expression in |
| | | | | prostate); CLL and | stomach, intestine, |
| | | | | diffuse large B-cell | spleen, lung, kidney, |
| | | | | lymphomas; melanoma | prostate, pancreas, |
| | | | | and nonmelanoma skin | and heart. |
| | | | | cancers; neuroblastoma | Transiently expressed |
| | | | | | in normal |
| | | | | | proliferating cells |
| | | | | | during the G2/M |
| | | | | | phase |
| Survivin- | A24 | AYACNTSTL | Hirohashi et al. [70] | The same as survivin | Thymus |
| 2b^{g} TRG | B52 | YQLCLTNIF^{h} | Ohkouchi et al. [128] | Breast, lung, colon, and | Low expression in |
| | B62 | | | prostate carcinomas | heart, liver, and |
| | | | | | pancreas |
| WTI | A2 | RMFPNAPYL | Oka et al. [132] | Gastric, colon, lung, | Kidney, ovary, testis, |
| | A24 | CMTWNQMNL | Obminami et al. | breast, ovary, uterine, | spleen |
| | | | [130] | thyroid, and | |
| | | RWPSCQKKF | Azuma et al. [9] | hepatocellular | |
| | | | | carcinomas; leukemia | |
| | | | | (including AML, ALL, | |
| | | | | and CML) | |
| 707-APⁱ | A2 | RVAALARDA | Morioka et al. [122] | Melanoma | None |

^{a}Unspliced transcript containing intron 2. The immunogenic peptide is entirely contained within the intronic sequence
^{b}The peptide is generated by a post-translational protein splicing
^{c}VLPDVFIRC(V) is the nonamer, and decamer peptides are both recognized by CTLs. The immunogenic peptide is entirely contained within the intronic sequence
^{d}Tissue distribution among tumors as described in the given references when different from the paper first reporting the sequence of the epitope
^{e}The immunogenic peptide is encoded by an alternative ORF
^{f}The epitope derives from mutated p53 protein, but does not contain the mutation
^{g}This is a splicing variant of survivin, retaining a part of intron 2 as a cryptic exon
^{h}The TRG gene is located in an intron of the putative tumor suppressor gene testin
ⁱThe immunogenic peptide sequence seems to be associated to an as-yet-unidentified antigen that is expressed in the majority of melanomas and in some tumors of other histological origin, but not in normal cells, as defined serologically [98]. However, as the tissue of the testis was not tested, it will not be clear to which category the antigen may belong until more information is available

TABLE 4

Class I HLA-restricted tumor-specific antigens, including
both unique and shared antigens. Underlined amino acids
in the epitopes indicate mutations or splicing aberration.
Normal tissues never express these epitopes. The table
does not include other tumor-specific antigens such as
fusion proteins, which are listed in Table 6

| | | | | |
|---|---|---|---|---|
| | HLA | Peptide | Tissue expression | |
| Gene | allele | epitope | in tumors | References |

Unique

| | | | | |
|---|---|---|---|---|
| α-Actinin-4 | A2 | FIASNGVKLV | Lung carcinoma | Echchakir et al. [49] |
| β-Catenin | A24 | SYLDSGIHF | Melanoma | Robbins et al. [148] |
| Caspase-8 | B35 | FPSDSWCYF | H/N tumor | Mandruzzato et al. [116] |
| CDK-4 | A2 | ACDPHSGHFV | Melanoma | Wölfel et al. [209] |
| ELF2 | A68 | ETVSEQSNV | Lung SCC | Hogan et al. [71] |
| HLA-A*0201- | A2 | CVEWLR IYLENGK | RCC | Brändle et al. [19] |
| R170I | | | | |
| HSP70-2 M | A2 | SLFEGIDIY | RCC | Gaudin et al. [55] |
| KIAA0205 | B44*03 | AEPINIQTV | Bladder cancer | Gueguen et al. [62] |
| Malic enzyme | A2 | FLDEFMEGV | SCC of the lung | Karanikas et al. [82] |
| MART-2 | A1 | FLEGNEVGKTY | Melanoma | Kawakami et al. [89] |
| MUM-1 | B44 | EEKLIVVLF | Melanoma | Coulie et al. [37] |
| MUM-2 | B44 | SELFRSGLDY | Melanoma | Chiari et al. [31] |
| | Cw6 | FRSGLDSYV | | |
| MUM-3 | A28 | EAFIQPITR | Melanoma | Baurain et al. [12] |
| Myosin | A3 | KINKNPKYK | Melanoma | Zorn and Hercend, 1999 |
| | | | | [219] |
| OS-9 | B44 | KELEGILLL | Melanoma | Vigneron et al. [192] |
| Shared | | | | |
| BING-4 | A2 | MCQWGRLWQL^{a} | Melanoma | Rosenberg et al. [153] |
| K-RAS | B35 | VVVGA VGVG | Pancreatic and colorectal | Gjertsen et al. [59] |
| | | | adenocarcinomas | |
| N-RAS | A1 | ILDTAG REEY | Melanoma | Linard et al. [109] |
| OGT | A2 | SLYKFSPFPL^{b} | Colon carcinomas (MSI⁺) | Ripberger et al. [146] |
| TGFαRII | A2 | RLSSCVPVVA^{b} | Colon carcinomas (MSI⁺) | Linnebacher et al. [110] |
| TRP-2/INT2 | A68 | EVISCKLIKR^{c} | Melanoma, glioblastoma | Lupetti et al. [114] |
| | | | multiforme [111] | |
| TRP-2-6b | A2 | ATTNILEHY^{d} | Melanoma, glioblastoma | Khong et al. [94] |
| | | | multiforme | |

^{a}The peptide derives from an altemative ORF
^{b}The peptide derives from a translational frameshift
^{c}The immunogenic peptide is entirely contained within the intronic sequence
^{d}The immunogenic peptide is encoded by exon 6b, one of the two novel exons alternatively spliced from intron 6

TABLE 5
Class II HLA-restricted antigens

| | | Peptide | Tissue expression | | |
|---|---|---|---|---|---|
| Gene | HLA allele | epitope | Tumors | Normal tissues | References |

(A) Epitopes from nonmutated protein antigens
Cancer-testis antigens

| | | | | | |
|---|---|---|---|---|---|
| CAMEL | DR11 | PWKRSWSA | The same as NY-ESO-1 | The same as NY-ESO-I | Slager |
| | DR12 | | (see below) | (see below) | et al. [166] |
| LAGE-1 | DRB1*1301 | ILSRDAAPLPRPG^{a} | The same as NY-ESO-1 | The same as NY-ESO-1 | Wang |
| | | | (see below) | (see below) | et al. [200] |
| MAGE-A1 | DRB1*1301 | LLKYRAREPVTKAE^{b} | Melanoma, myeloma | Testis, placenta | Chaux |
| | DRB1*1302 | | (stage III), lung | | et al. [28] |
| | | | carcinoma, H/N SCC, | | |
| | | | esophageal SCC, | | |
| | | | superficial and | | |
| | | | infiltrating bladder | | |
| | | | carcinoma | | |
| MAGE-A2 | DRB1*1301 | LLKYRAREPVTKAE^{b} | The same as MAGE-A1 | The same as MACE-A1 | Chaux |
| | DRB1*1302 | | | | et al. [28] |
| MAGE-A3 | DRB1*1101 | TSYVKVLHHMVKISG | The same as MAGE-A1 | The same as MAGE-A1 | Manici |
| | | | | | et al. [117] |
| | DRB1*1301, | LLKYRAREPVTKAE^{b} | | | Chaux |
| | DRB1*1302 | | | | et al. [28] |
| | DRB1*1301, | AELVHFLLLKYRAR^{b} | Melanoma, lung and | Testis, placenta | Chaux |
| | DRB1*1302 | | breast carcinomas, | | et al. [29] |
| | | | H/N SCC | | |
| | DR1, DR4, | RKVAELVHFLLLKYR^{b} | Melanoma, lung and | Testis, placenta | Consogno |
| | DR11^{c} | GDNQIMPKAGLLIIV | breast carcinomas, | | et al. [33] |
| | | TSYVKVLHHMVKISG | H/N SCC | | |
| | DR1, DR4, | FFPVIFSKASSSLQL^{b} | | | Consogno |
| | DR7, DR11^{c} | | | | et al. [33] |
| MAGE-A6 | DRB1*1301, | LLKYRAREPVTKAE^{b} | The same as MAGE-A1 | The same as MAGE-A1 | Chaux |
| | DRB1*1302 | | | | et al. [28] |
| | DRB1*0401 | ESEFQAALSRKVAKL, | | | Tatsumi |
| | | LLKYRAREPVTKA- | | | et al. [172] |
| | | EMLGSVVGNWQ, | | | |
| | | VGNWQYFFPVIFSKA- | | | |
| | | SDSLQLVFGIELMEVD, | | | |
| | | IFSKASDSLQLVFGIE, | | | |
| | | LTQYFVQENYLEYRQVPG | | | |
| NY-ESO-1 | DRB4*0101 | VLLKEFTVSG | Melanoma; myeloma | Testis, placenta | Zeng |
| DRB4*0101- | PLPVPGVLLKEFTVSGNI | (stage III); lung | (very low levels) | et al. [217] | |
| 0103 | VLLKEFTVSGNILTIRLT | carcinoma; H/N SCC; | | Jager | |
| | | AADHRQLQLSISSCLQQL | esophageal SCC; | | et al. [78] |
| | | | infiltrating bladder, | | |
| | | | prostate, and breast | | |
| | | | carcinomas | | |

Differentiation antigens

| | | | | | |
|---|---|---|---|---|---|
| CEA | DR9 | YACFVSNLATGRNNS | Overexpressed in | Epithelial | Kobayashi |
| | DR*03, | LWWVNNQSLPVSP | colon carcinoma and | differentiation | et al. [103] |
| | DR*0405, | | other adenocarcinomas | antigen | Campi |
| | DR*07, | | | | et al. [24] |
| | DR*1101, | | | | |
| | DR*1104, | | | | |
| | DR*14^{c} | | | | |
| Gp100 | DRB1*0401 | WNRQLYPEWTEAQRLD | Melanoma | Melanocytes | Li |
| | | | | | et al. [108] |
| | DRB1*0701 | TGRAMLGTHTMEVTVYH | | | Lapointe |
| | | | | | et al. [106] |
| | DRB1*0401 | IYRRRLMKQDFSVPQLPHS | | | Kierstead |
| | | | | | et al. [96] |
| MART-1/ | DRB1*0401 | RNGYRALMDKSLHVGTQ- | Melanoma | Melanocytes | Zarour |
| Melan-A | | CALTRR | | | et al. [216] |
| PSA | DRB1*0401 | ILLGRMSLFMPEDTG | Melanoma | Melanocytes, | Corman |
| | | SLFHPEDTGQVFQ | | prostate, gland | et al. [34] |
| | | QVFQVSHSFPHPLYD | | | |
| | | NDLMLLRLSEPAELT | | | |
| | | KKLQCVQLHVISM | | | |
| | | GVLQGITSMGSEPCA | | | |
| Tyrosinase | DRB1*0401 | QNILLSNAPLGPQFP | Melanoma | Melanocytes | Topalian |
| | | DYSYLQDSDPDSFQD | | | et al. [176] |
| | | SYLQDSDPDSFQD | | | Topalian |
| | | | | | et al. [177] |
| | DRB1*1501 | RHRPLQEVYPEANAPIGHNR | | | Kobayashi |
| | | | | | et al. [101] |
| | DRB1*0405 | E | | | Kobayashi |
| | | | | | et al. [102] |
| | DRB1*0401 | EIWRDIDFAHE | | | Kierstead |
| | | YGQMKNGSTPMFNDIMYDL | | | et al. [96] |
| | | ALHIYMDGTMSQVQGSA | | | |

Widely expressed antigens

| | | | | | |
|---|---|---|---|---|---|
| Annexin II | DRB1*0401 | DVPKWISIMTERSVPH | Melanoma | Endothelial, | Li |
| | | | | mesothelial, and | et al. [108] |
| | | | | some epithelial | |
| | | | | cells; peripheral | |
| | | | | nerves; part of | |
| EphA3 | DRB1*1101 | DVTFNIICKKCG | Overexpressed in | High expression in | Chiari |
| | | | melanoma, SC and | retina and in fetal | et al. [32] |
| | | | NSCLC, sarcomas, and | brain. Significant | |
| | | | RCC | expression in bladder, | |
| | | | | prostate, and colon | |
| | | | | Low expression in | |
| | | | | several other normal | |
| | | | | tissues but | |
| | | | | hematopoietic cells. | |
| | | | | Melanocytes do not | |
| | | | | express the protein | |
| HER-2/neu | DR11 | GSYVSRLLGICL | Melanoma; ovarian, | Epithelial cells | Anderson |
| | | VPIKWMALESILRRRF | gastric, pancreatic | | et al. [5] |
| | | | [141], and breast | | |
| | | | carcinomas | | |
| MUC1 | DR3 | PGSTAPPAHGVT | Breast and ovarian | None^{d} | Hiltbold |
| | | | cancers; multiple | | et al. [69] |
| | | | myeloma; B-cell | | |
| | | | lymphoma | | |
| WT1 | DRB1*0401 | PQQMGSDVRDLNALL | Gastric, colon, | Kidney, ovary, | Knights |
| | | | lung, breast, ovary | testis, spleen | et al. [100] |
| | | | uterine, thyroid, and | | |
| | | | hepatocellular | | |
| | | | carcinomas; leukemia | | |
| | | | (including AML, ALL, | | |
| | | | and CML) | | |

(B) Epitopes from mutated protein antigens. Underlined are the mutated amino acids and the peptide
sequences deriving from mutations or splicing aberration

Unique

| | | | | | |
|---|---|---|---|---|---|
| CDC27 | DRB1*0401 | FSWAMDLDPKGA^{e} | Melanoma | None | Wang |
| | | | | | et al. [205] |
| FN | DR2 | MIFEKHGFRRTTPP | Melanoma | None | Wang |
| | | | | | et al. [199] |
| Neo-PAP | DR7 | RVIKNSIRLTL^{e} | Melanoma | None | Topalian |
| | | | | | et al. [178] |
| PTPRK | DRB1*1001 | PYYFAAELPP RNLPEP | Melanoma | None | Novellino |
| | | | | | et al. [127] |
| TPI | DRB1*0101 | GELIG/LNAAKVPAD | Melanoma | None | Pieper |
| | | | | | et al. [143] |
| Shared | DR (not | SLVRLSSCVPVALMSA- | Colon carcinomas | None | Saeterdal |
| TGFβRII | identified) | MTTSSSQ^{f} | (MSI⁺) | | et al. [155] |

^{a}This epitope is specifically recogized by CD4⁺ T-regulatory cells that were cloned by limiting dilution from TLLs deriving from a fresh melanoma sample. These cells significantly suppressed autorogous effector CD4⁺ T cells following a LAGE epitope ligand-specific activation
^{b}These epitopes share different HLA-DR due to the known promiscuity of peptide binding to HLA-DR molecules. This allows an epitope to be potentially used for cancer immunotherapy in a larger number of patients
^{c}In the paper, not all the HLA-DR alleles were completely subtyped
^{d}All epithelial tissues express highly glycosylated mucins, whereas tumor cells often show hypoglycosylated mucins with a normal protein sequence
^{e}The mutation is not located in the region encoding the peptide
^{f}The peptide derives from a translational frameshift

TABLE 6

Epitopes derived from chimeric proteins originated
by gene translocation and fusion processes that do
not normally occur in normal tissues. Therefore,
these antigens are tumor-specific. Underlined are
the sequences after the junction point

| | | | | |
|---|---|---|---|---|
| | | | Tissue | |
| | | Peptide | distribution | |
| Gene | HLA allele | epitope | among tumors | References |

HLA class I-restricted epitopes

| | | | | |
|---|---|---|---|---|
| abl-bcr alb-b3 (b2a2) | A*0201 | FVEHDDESPGL | CML | Wagner et al. [198] |
| abl-bcr alb-b4 (b3a2) | A*0201 | FVEHDLYCTL | CML | Wagner et al. [198] |
| bcr-abl^{a} | A2 | FMVELVEGA | CML | Buzyn et al. [23] |
| | | KLSEQESLL | | |
| | | MLTNSCVKL | | |
| bcr-abl p210 (b3a2) | A2 | SSKALQRPV | CML | Yatuda et al. [213] |
| | A3 | ATGFKQSSK | | Greco et al. [61] |
| | | KQSSKALQR | | |
| | A3, A11 | HSATGFKQSSK | | Bocchia et al. [15] |
| | A3 | KQSSKALQR | | Norbury et al. [126] |
| | B8 | GFKQSSKAL | | Norbury et al. [126] |
| ETV6/AML | A2 | RIAECILGM | ALL | Yotnda et al. [214] |
| NPM/ALK^{b} | A2*0201 | SLAMLDLLHV | NPM/ALK: in | Passoni et al. [139] |
| | | | anaplastic large | |
| | | | cell lymphomas | |
| | | GVLLWEIFSL | ALK: in | |
| | | | neuroblastomas | |
| SYT/SSX | B7, B42 | QRPYGYDQIM | Synovial | Worley et al. [210] |
| | | | sarcoma | |

HLA class II-restricted epitopes

| | | | | |
|---|---|---|---|---|
| abl-bcr alb-b3 (b2a2) | DRB1*0701 | GPHCNVFVEHDDESPGLYG | CML | Wagner et al. [198] |
| bcr-abl p190 (ela2) | DRB1*1501 | EGAFHGDAEALQRPVAS | ALL | Tanaka et al. [170] |
| bcr-abl p210 (b2a2) | DRB5*0101 | IPLTINKEEALQRPVAS | CML | ten Bosch et al. [175] |
| bcr-abl p210 (b3a2) | DRB1*0401 | ATGFKQSSKALQRPVAS^{c} | CML | ten Bosch et al. [174] |
| | DRB1*1501 | ATGFKQSS KALQRPVAS^{c} | | ten Bosch et al. [173] |
| | DRB1*0901 | ATGFKQSS KALQRPVAS^{c} | | Yasukawa et al. [212] |
| | DRB1*1101 | LIVVIVHSATGFKQSS | | Pawelec et al. [140] |
| | | KALQRPVA | | |
| | DR11 | IVHSATGFKQSS | | Bocchia et al. [15] |
| | | KALQRPVASDFEP | | |
| DEK-CAN | DRB4*0103 | TMKQICKK EIRRLHQY | AML | Obminami et al. [129] |
| LDLR/FU7^{d} | DRBI*0101 | GGAPPVTWRRAPAPG | Melanoma | Wang et al. [206] |
| | | WRRAPAPGAKAMAPG | | |
| pml/RARα | DR11 | NSNHVASGAGEAAIETQSSSS | APL | Gambacorti-Passerini |
| | | EEIV [43] | | et al. [54] |
| TEL/AMLI | DP5, DP17 | IGRIAECILGMNPSR | AML | Yun et al. [215] |

^{a}These bcr-abl epitopes derive from the BCR part of the chimeric protein and do not span the fusion junction. BCR is ubiquitously expressed in normal cells. From an immunotherapeutic point of view these peptides could be considered as widely/overexpressed epitopes rather than as tumor-specific fusion protein-derived epitopes
^{b}The two epitopes occur entirely within the ALK region of the antigen, and do not span the fusion junction. CTLs directed against these two epitopes recognize both NPM/ALK⁺ lymphomas and ALK⁺ neuroblastomas. The ALK protein is normally expressed only in pericytes and scattered glial cells of selected regions of the CNS, such as the hypothalamus
^{c}These epitopes share different HLA-DR alleles. This allows an epitope to be employed for cancer immunotherapy in a larger number of patients
^{d}The antigen is unique to the melanoma patient examined, and the epitopes do not span the junction region. However, the fusion between the two proteins does generate the epitopes, as they derive from the antisense translation of the FUT sequence of the fusion protein

TABLE 7
Frequency of epitopes recognized by a given HLA allele. In the case of cancer-testis and melanoma
differentiation groups, the TAAs most frequently used in clinical trials are outlined

| | No. of | | | | | | | | |
|---|---|---|---|---|---|---|---|---|---|
| TAA | epitopes | HLA-A | % | HLA-B | % | HLA-C | % | HLA-DR | % |
| Cancer-testis | | | | | | | | | |
| MAGE-1, -2, -3, -4, -6, -10, -12 | 42 | 14 | 33.3 | 9 | 21.4 | 4 | 9.5 | 15 | 35.7 |
| GAGE-1, -2, -3, -4, -5, -6, -7B, -8 | 2 | 1 | 50 | 0 | | 1 | 50 | 0 | |
| NY-ESO-1 | 9 | 4 | 44.4 | 1 | 11.1 | 0 | | 4 | 44.4 |
| Other cancer-testis antigens | 11 | 6 | 54.5 | 1 | 9.1 | 2 | 18.2 | 2 | 18.2 |
| Melanoma differentiation | | | | | | | | | |
| Gp100 | 21 | 16 | 76.2 | 1 | 4.8 | 1 | 4.8 | 3 | 14.3 |
| MART-1/Melan-A | 7 | 3 | 42.8 | 3 | 42.8 | 0 | | 1 | 14.3 |
| Tyrosinase | 14 | 5 | 35.7 | 2 | 14.3 | 0 | | 7 | 50 |
| Other melanoma and nonmelanoma | 28 | 19 | 67.8 | 0 | | 1 | 3.6 | 8 | 28.6 |
| differentiation antigens | | | | | | | | | |
| Widely expressed | 71 | 59 | 83.1 | 7 | 9.8 | 0 | | 5 | 7 |
| Unique and shared tumor-specific | 28 | 15 | 53.6 | 6 | 21.4 | 1 | 3.6 | 6 | 21.4 |
| Fusion protein | 28 | 13 | 46.4 | 2 | 7.2 | 0 | | 13 | 46.4 |

Examples of viral antigens that may be used in the practice of this invention include, without limitation, those derived from: herpes simplex virus (HSV), hepatitis B virus (HBV), hepatitis C virus (HCV), cytomegalovirus (CMV), Epstein-Barr virus (EBV), influenza virus, measles virus, human immunodeficiency virus (HIV), and human papilloma virus (HPV).

In one embodiment, the antigen used may be a cellular fraction enriched for molecules containing a carbohydrate moiety, such as glycoproteins or glycolipids. Methods for enriching for cellular components containing carbohydrate moieties are well known in the art, including by lectin binding and the like.

It will be appreciated by the skilled artisan that the antigens used in the practice of the invention can be covalently or noncovalently associated with the heat shock protein cages of the invention. If covalent association is used, chemical linkers such as those disclosed herein can be used to join the antigen to the inside or outside of heat shock protein cages. For chemical linkage, heat shock protein subunits modified to contain reactive groups suitable for coupling, such as cysteine or lysine residues, can be used. Alternatively, recombinant methods may be used to create fusion proteins between a heat shock protein and an antigen. When such methods are used, the antigen can be either on the inside or outside of heat shock protein cages, depending on whether the fusion is made at the N- or C-terminus of the heat shock protein subunit.

C. Immunological Adjuvants

In some embodiments, the Hsp protein cages of the invention can include immunological adjuvants. As used herein, an adjuvant is an agent which, while not having any specific antigenic effect itself, can stimulate the immune system, increasing the response to a vaccine. Many adjuvants accomplish this task by mimicking specific sets of evolutionarily conserved molecules including lipopolysaccharide (LPS), components of bacterial cell walls (e.g., Klebsiella kpOmpA), and endocytosed nucleic acids such as double-stranded RNA (dsRNA), single-stranded DNA (ssDNA), and unmethylated CpG dinucleotide-containing DNA (see, Gavin et al., Science, 314: 1936-8 (2006)). Because immune systems have evolved to recognize these specific antigenic moieties, the presence of adjuvant with vaccine can greatly increase the innate immune responses to antigen by augmenting the activities of dendritic cells (DCs), lymphocytes and macrophages. Accordingly, among the adjuvants that may be used in the practice of this invention include: lipid A, muramyl di-peptide (MDP), CpG motifs, or polyI/polyC, endotoxin, lipopolysaccharide (LPS), kpOmpA, and aluminum salts.

As discussed herein, an adjuvant, if used in the practice of this invention, can be attached covalently or noncovalently to the interior or exterior of an Hsp protein cage. In the case of adjuvant reagents that may be labile, for instance, under physiological conditions or easily degraded by enzymes, adjuvant can advantageously be positioned on the interior of Hsp protein cages to provide some protection from the effects of solvents and other agents on the exterior protein cages.

It will be appreciated by the skilled artisan that adjuvant can also be used in a formulation of heat shock protein cages for administration to a subject. In such an instance, the heat shock protein cages will be formulated in a preparation of adjuvant using methods known in the art. The heat shock protein cages for such applications can contain the same, or different, or no, adjuvant as that used to make up the formulation for administration.

D. Modifications of Hsp Protein Cages

As will be appreciated by those in the art, the monomers of the protein cages can be naturally occurring or variant forms, including amino acid substitutions, insertions and deletions (e.g. fragments) that can be made for a variety of reasons as further outlined below. For example, amino acid residues on the outer surface of one or more of the monomers can be altered to facilitate functionalization for attachment to additional moieties (targeting moieties such as antibodies, polymers for delivery, the formation of noncovalent chimeras), to allow for crosslinking (e.g., the incorporation of cysteine residues to form disulfides). Similarly, amino acid residues on the internal surfaces of the shell can be altered to facilitate payload molecule loading, stability, to create functional groups which may be later modified by the chemical attachment of other materials (small molecules, polymers, proteins, etc.).

With respect to some embodiments, in particular, those with dodecameric protein cages, the natural channels to the interior formed by the two-, three-, and four-fold symmetry of the dodecameric proteins may be modified to enable either the introduction and/or extraction, or both, of materials through the opening therein.

It will be appreciated that covalent modifications of protein cages are included within the scope of this invention. One type of covalent modification includes reacting targeted amino acid residues of a cage residue with an organic derivatizing agent that is capable of reacting with selected side chains or the N- or C-terminal residues of a cage polypeptide. Derivatization with bifunctional agents is useful, for instance, for crosslinking the cage to a water-insoluble support matrix or surface for use in the methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl propionate), bifunctional maleimides such as bis-N-maleimido-1,8-octane and agents such as methyl-3-[(p-azidophenyl)dithio]propioimidate.

Alternatively, functional groups can be added to the protein cage for subsequent attachment to additional moieties. Preferred functional groups for attachment are amino groups, carboxy groups, oxo groups, and thiol groups. These functional groups can then be attached, either directly or indirectly through the use of a linker. Linkers are well known in the art; for example, homo- or hetero-bifunctional linkers as are well known (see, 1994 Pierce Chemical Company catalog, technical section on cross-linkers, pages 155-200, as well as the 2003 catalog, both of which are incorporated herein by reference). Preferred linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred. In some cases, the linkers are cleavable by conditions such as alkali, acid, reduction, oxidation, protease, nuclease or electromagnetic radiation, or heat treatment. See, e.g., Flenniken et al., Chem. Comm., 447-449 (2005); Willner et al., Bioconj. Chem., 4:521-7 (1993); U.S. Pat. Nos. 5,767,288 and 4,469,774.

In some embodiments, linkers that influence some property of an attached protein, such as folding, net charge, or hydrophobicity can be used. Other linkers include ones that are cleavable by conditions at the site of action of the payload, such as the pH of a particular cellular compartment, or the presence of a protease. Accordingly, such linkers can be used to attach payload molecules to the interior of protein cages. In some embodiments, the linkers will contain sequences that are cleavable by enzymes or conditions in a cell or tissue targeted by the targeting moiety. This feature of the linkers will allow for the controlled release of covalently attached antigens or adjuvant at a specific site, such as within an APC.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of cages, if appropriate, comprises altering the native glycosylation pattern of the polypeptide. “Altering the native glycosylation pattern” is intended to generally include deleting one or more carbohydrate moieties found in the native sequence of the cage monomer, and/or adding one or more glycosylation sites that are not present in the native sequence.

Yet another type of covalent modification is to synthesize protein cages with nonnatural amino acids that have unique points of conjugation. To effect such modifications, amber codon suppression mutagenesis is used to introduce nonnatural amino acids in a site specific manner. The incorporation of nonnatural amino acids bearing ketones, azides or alkynes into proteins has been accomplished using this methodology. Such modifications allow further derivatization using hydrozone formation, Staudinger ligation or azide/alkyne cycloaddition reactions, among others. Use of this type of covalent modification allows for specific spatial placement of targeting moieties and controlled stoichiometry. See, Chen et al., Current Opinion in Biotechnology, 16:35-40 (2005), for review; see, also, Wang et al., Annu. Rev. Biophys. Biomol. Struct., 35:225-49 (2006); Chin et al., J. Am. Chem. Soc., 124:9026-9027 (2002).

Addition of glycosylation sites to cage polypeptides may be accomplished by altering the amino acid sequence thereof. The alteration can be made, for example, by the addition of, or substitution by, one or more serine or threonine residues to the native sequence polypeptide (for O-linked glycosylation sites). The amino acid sequence can optionally be altered through changes at the DNA level, particularly by mutating the DNA encoding the polypeptide at preselected bases such that codons are generated that will translate into the desired amino acids.

Another means of increasing the number of carbohydrate moieties on the polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of cage moieties comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337. This finds particular use in increasing the physiological half-life of the composition.

Hsp-cage polypeptides of the present invention can also be modified in a way to form chimeric molecules comprising a cage polypeptide fused to another, heterologous polypeptide or amino acid sequence. In one embodiment, such a chimeric molecule comprises a fusion of a cage polypeptide with a tag polypeptide, which provides an epitope to which an antitag antibody can selectively bind. The epitope tag is generally placed at the amino- or carboxyl-terminus of the Hsp polypeptide. The presence of such epitope-tagged forms of a cage polypeptide can be detected using an antibody against the tag polypeptide. Also, provision of the epitope tag enables the cage polypeptide to be readily purified by affinity purification using an antitag antibody or another type of affinity matrix that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In a preferred embodiment, the protein cages are derivatized for attachment to a variety of moieties, including but not limited to, dendrimer structures, additional proteins, carbohydrates, lipids, targeting moieties, and the like. In general, one or more of the subunits is modified on an external surface to contain additional moieties.

In a preferred embodiment, the protein cages can be derivatized as outlined herein for attachment to polymers. The character of the polymer will vary, but in certain embodiments, the polymer either contains, or can be modified to contain functional groups for the attachment of the protein cages of the invention. Suitable polymers include, but are not limited to, functionalized dextrans, styrene polymers, polyethylene and derivatives, polyanions including, but not limited to, polymers of heparin, polygalacturonic acid, mucin, nucleic acids and their analogs including those with modified ribose-phosphate backbones, the polypeptides polyglutamate and polyaspartate, as well as carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and polypeptides such as protamine, the histone polypeptides, polylysine, polyarginine and polyornithine; and mixtures and derivatives of these. Particularly preferred polycations are polylysine and spermidine. Both optical isomers of polylysine can be used. The D isomer has the advantage of having long-term resistance to cellular proteases. The L isomer has the advantage of being more rapidly cleared from an animal when administered. As will be appreciated by those in the art, linear and branched polymers may be used.

A preferred polymer is polylysine, as the —NH₂ groups of the lysine side chains at high pH serve as strong nucleophiles for multiple attachment of ligands (e.g., antigens, adjuvants, targeting moieties, etc.) to protein cages.

The size of the polymer may vary substantially. For example, it is known that some nucleic acid vectors can deliver genes up to 100 kilobases in length, and artificial chromosomes (megabases) have been delivered to yeast. Therefore, there is no general size limit to the polymer. However, a preferred size for the polymer is from about 10 to about 50,000 monomer units, with from about 2000 to about 5000 being particularly preferred, and from about 3 to about 25 being especially preferred.

E. Targeting Moieties

The present invention also optionally provides targeting moieties that direct Hsp protein cages to specific molecular and cellular sites. A “targeting moiety” refers to a functional group which serves to target or direct the Hsp protein cage complex to a particular location, site, cell type, or molecular association. In general, the targeting moiety is directed against and binds a target molecule and allows the accumulation of the compositions to a particular location, for instance, to a particular cell type, tissue, or anatomical location within a subject. Thus, for example, antibodies, cell surface receptor ligands and hormones, lipids, sugars and dextrans, alcohols, bile acids, fatty acids, sterols, amino acids, peptides and nucleic acids may all be attached to Hsp protein cages to localize or these compositions to a particular site. In one embodiment, the composition is partitioned to the location in a non-1:1 ratio. Particularly advantageous targeting moieties are those that target Hsp protein cages to APC's. Among the cell surface molecules on APC's that may serve as targets are: the Fc receptor, clathrin coated pit proteins, chemokine receptors, and cytokine receptors.

Examples of targeted protein cages comprising a variety of targeting moieties may be found in U.S. patent application Ser. No. 12/035,928, PCT/US08/54,745, and PCT/US08/59,238, which are incorporated by reference herein in their entireties.

An example of an especially advantageous targeting moiety is an antibody. The term “antibody” generally includes an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See, also, Pierce Catalog and Handbook, 1994-1995 (Pierce Chemical Co., Rockford, Ill.). See, also, e.g., Kuby, Immunology, 3.sup.rd Ed., W. H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al., J. Immunol., 148:1547 (1992); Pack and Pluckthun, Biochemistry, 31:1579 (1992); Hollinger et al., supra; Gruber et al., J. Immunol, 152:5368 (1994); Zhu et al., Protein Sci., 6:781 (1997); Hu et al., Cancer Res., 56:3055 (1996); Adams et al., Cancer Res., 53:4026 (1993); and McCartney et al., Protein Eng., 8:301 (1995).

An antibody immunologically reactive with a particular antigen can be generated by recombinant methods such as selection of libraries of recombinant antibodies in phage or similar vectors, see, e.g., Huse et al., Science, 246:1275-1281 (1989); Ward et al., Nature, 341:544-546 (1989); and Vaughan et al., Nature Biotech., 14:309-314 (1996), or by immunizing an animal with the antigen or with DNA encoding the antigen.

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies can, alternatively, be monoclonal antibodies. Monoclonal antibodies may be prepared using hybridoma methods, such as those described by Kohler & Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. Generally, either peripheral blood lymphocytes (“PBLs”) are used if cells of human origin are desired, or spleen cells or lymph node cells are used if nonhuman mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Human antibodies can be produced using various techniques known in the art, including phage display libraries (Hoogenboom & Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol., 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., BioTechnology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); Lonberg & Huszar, Inter. Rev. Immunol., 13:65-93 (1995).

F. Other Components of Hsp Protein Cages

It will be appreciated that in some instances it will be desirable to include other reagents in association with the Hsp protein cages of the invention, other than adjuvant, which augment the immunological effect of an antigen. For example, nucleic acid reagents that alter the expression of genes within a target cell, such as an APC, may be advantageously included within the heat shock protein cages of the present invention. Examples of such nucleic agent reagents that have been used with APC's include siRNAs. For example, siRNAs have been used to reduce the expression of the suppressor of cytokine signaling 1 (SOCS1) gene in dendritic cells. See, Mao et al., J. Biomed. Sci., 14:15-29 (2007); et al., Gene Therapy, 13:1714-1723 (2006).

Of particular interest for inclusion with heat shock protein cages are nucleic acids such as antisense nucleic acids, siRNAs, or ribozymes that are able to inhibit the expression of specific genes.

Antisense nucleic acids fall into the categories of enzyme-dependent antisense or steric blocking antisense. Enzyme-dependent antisense includes forms dependent on RNase H activity to degrade a target mRNA, including single-stranded DNA, RNA, and phosphorothioate antisense. Double stranded RNA acts as enzyme-dependent antisense through the RNAi/siRNA pathway, involving target mRNA recognition through sense-antisense strand pairing followed by target mRNA degradation by the RNA-induced silencing complex (RISC). Steric blocking antisense (RNase-H independent antisense) interferes with gene expression or other mRNA-dependent cellular processes by binding to a target sequence of mRNA and getting in the way of other processes. Steric blocking antisense includes 2′-O alkyl (usually in chimeras with RNase-H dependent antisense), peptide nucleic acid (PNA), locked nucleic acid (LNA) and Morpholino antisense.

Small interfering RNA (siRNA), sometimes known as short interfering RNA or silencing RNA, are a class of 20-25 nucleotide-long double-stranded RNA molecules that are involved in the RNA interference (RNAi) pathway by which the siRNA interferes with the expression of a specific gene. Generally, siRNAs are short (usually 21-nt) doubled-stranded RNAs (dsRNAs) with 2-nt 3′ overhangs on either end. See, generally, Hannon et al., Nature, 431:371-378 (2004).

Ribozymes that cleave mRNA at site-specific recognition sequences are used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene-targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Other means of modifying or blocking particular functions in APC's to enhance an immune response are also known. For example, the use of anti-CTLA antibodies to block this protein was found to result in a durable antitumor T cell response when mice were provided with a peptide vaccine in combination with a CpG adjuvant (see Davila et al., Cancer Res. (2003) 63: 3281-8). Reagents such as anti-CTLA, or other blocking or activating antibodies, may be co-administered with or attached to the Hsp protein cages of the present invention.

Alternatively, nucleic acids that are capable of expressing a sequence encoding an antigen of interest can be included within the heat shock protein cages. Such nucleic acids can, for example, be delivered to APC's, resulting in the expression of an antigen of interest within the APC. The resulting protein can then be processed for the presentation of peptides on the cell surface by MHC molecules. For such uses, expression constructs comprising the gene of interest under the control of an appropriate may be generated using molecular biological methods well known in the art.

G. Chimeric Hsp Protein Cages

It will be appreciated that the generation of chimeric Hsp protein cages are also contemplated by the present invention. Thus, for instance, a Hsp protein cage can comprise multiple different antigens attached to heat shock protein subunits. For example, such a chimeric Hsp protein cage may be derived by: (1) generating a Hsp-antigen-A fusion protein and a Hsp-antigen-B fusion protein; (2) disrupting the protein cages that result from each respect fusion protein into monomers; and (3) mixing the Hsp-antigen-A and -B monomers together under conditions under which protein cages will re-form. Alternatively, the Hsp-antigen-A and -B fusion proteins can be co-expressed in a single host cell with the expectation that chimeric Hsp-antigen-A and -B protein cages will form. It will be appreciated that protein cages chimeric for other components, such as adjuvant or targeting moieties, can be generated by similar means (e.g., using a linker to join the Hsp subunit to an adjuvant molecule).

### III. Formulations and Methods of Administration

Pharmaceutically acceptable carriers useful for the practice of this invention are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can consist of (a) liquid solutions, such as an effective amount of the compound suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The composition of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the compound with a suppository base. Suitable suppository bases include, but are not limited to, natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

An therapeutically effective dosage or amount of the Hsp protein cages of the present invention as therapeutic vaccines to elicit specific immune responses to Hsp proteins, or to substances conjugated to the Hsp proteins, such as antigenic proteins or peptides, is in the range of 0.1 to 2000 μg Hsp per injection, depending on the individual to whom the Hsp protein is being administered (see, e.g., Lussow et al., Eur. J. Immun., 21:2297-2302 (1991); Barrios et al., Eur. J. Immun., 22:1365-1372 (1992)). The appropriate dosage of the Hsp protein cages for each individual will be determined by taking into consideration, for example, the particular Hsp protein being administered, the nature of the antigen, whether an adjuvant is employed, the type of individual to whom the Hsp protein cage is being administered, the age and size of the individual, the condition being treated or prevented and the severity of the condition. The appropriate dosage to administer to an individual can be determined by the skilled artisan using no more than routine experimentation.

The pharmaceutical preparations are typically delivered to a mammal, including humans and nonhuman mammals. Nonhuman mammals treated using the present methods include domesticated animals (i.e., canine, feline, murine, rodentia, and lagomorpha) and agricultural animals (bovine, equine, ovine, porcine).

### IV. Examples

The following examples are offered to illustrate, but not to limit, the claimed invention.

It has been previously shown that human and animal immune systems will respond to antigens attached to soluble heat shock proteins to generate cellular immunity and thereby prevent from becoming established, eradicate existing, and prevent recurrences of dysplastic tissue or tumors expressing those antigens.

A peptide or protein to which one wants to raise cellular immunity in a human or animal in need of therapy is incorporated into a ProteoCage composed of the small heat shock protein of M. jannaschii. The peptide or protein can be entrapped (not chemically attached) to the inside of the Hsp ProteoCage; it can also be covalently attached via a chemical linker directly to the ProteoCage Hsp or by means of a longer chain linker composed of various polymers including ones composed of amino acids, carbohydrates or synthetic entities. The linkers can be designed to break under various conditions such as lowered pH or by the action of intracellular enzymes such as proteases. The antigenic peptide may also be chemically attached to the exterior surface of the Hsp ProteoCage.

An adjuvant, including ones such as unmethylated polynucleotide sequences such as CpG's and their derivatives, or polynucleotides such as double stranded poly IC, can also be entrapped or covalently attached within the Hsp ProteoCage. (See, e.g., Singh et al., Pharm. Res., 19:715-28 (2002); O'Hagan et al., Biomol. Eng., 18:69-85 (2001) for reviews.)

Alternatively, the adjuvant may be on the outside of the Hsp ProteoCage. The adjuvant can be covalently attached or the Hsp ProteoCage may be formulated in a solution or other preparation in which the adjuvant is dissolved or suspended in the solution in which the Hsp ProteoCage is also dissolved or suspended.

### Example 1

Human papilloma Virus Type 16 (HPV 16) is associated with and is considered to pose a high risk for development of cervical dysplasia and cervical cancer in women and anal dysplasia and anal cancer in women and men. The E7 protein from HPV 16 is an oncogene which is known to be a transforming agent and is known to be expressed in precancerous and cancerous lesions caused by its continual presence. Once appropriately stimulated, the immune system of a human with such virally induced lesions should be able to eradicate cells expressing antigenic peptides derived from this protein.

To construct a therapeutic vaccine, one that will activate and stimulate the immune system to attack and destroy already existing lesions, the HPV 16 E7 is entrapped in a ProteoCage composed of the small heat shock protein of M. jannaschii. The E7 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT.

A murine animal model is employed to demonstrate the appropriate antiviral and antitumor activity in vivo. The TC-1 cell line was created from epithelial cells transformed with HPV E6 and E7 oncogenes and also activated h-RAS. The cell line expresses antigens derived from these proteins. When injected subcutaneously in the flank of a mouse, the cells grow to produce a nonmetastatic tumor that, when left untreated, overwhelms the mouse, killing it. The presence or absence of the tumor is easily measured by palpation and the size of the tumor is measured using appropriately sized calipers. When microgram quantities of the ProteoCage containing the HPV 16 E7 are injected subcutaneously under the scruff of the neck of a mouse that has a measurable tumor growing in the distal back of the animal, the tumor will begin to shrink. After 14 to 21 days, the tumor will have completely disappeared. Histopathology of the former tumor site reveals the complete absence of tumor cells. Implantation of additional tumor cells into another region in the back of the cured mouse will result in the rejection of the new tumor cells and prevent the outgrowth of a new tumor, suggesting that the ProteoCage preparation produced long term memory capable of preventing recurrences. A similar result is expected when the antigen is covalently attached to the Hsp ProteoCage.

### Example 2

Human papilloma Virus Type 16 (HPV 16) is associated with and is considered high risk to cause cervical dysplasia and cervical cancer in women and anal dysplasia and anal cancer in women and men. The E6 protein from HPV 16 is an oncogene which is known to be a transforming agent and is known to be expressed in precancerous and cancerous lesions caused by its continual presence. Once appropriately stimulated, the immune system of the human with such virally induced lesions should be able to eradicate cells expressing antigenic peptides derived from this protein.

To construct a therapeutic vaccine, one that will activate and stimulate the immune system to attack and destroy already existing lesions, the HPV 16 E6 is entrapped in a Hsp ProteoCage composed of the small heat shock protein of M. jannaschii. The E6 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT.

A murine animal model is employed to demonstrate the appropriate antiviral and antitumor activity in vivo. The TC-1 cell line was created from epithelial cells transformed with HPV E6 and E7 oncogenes and also activated h-RAS. The cell line expresses antigens derived from these proteins. When injected subcutaneously in the distal back of a mouse, the cells grow to produce a nonmetastatic tumor that, when left untreated, overwhelms the mouse, killing it. The presence or absence of the tumor is easily measured by palpation and the size of the tumor is measured using appropriately sized calipers. When microgram quantities of the Hsp ProteoCage containing the HPV 16 E6 are injected subcutaneously under the scruff of the neck of a mouse that has a measurable tumor growing in the distal back of the animal, the tumor will begin to shrink. After 14 to 21 days, the tumor will have completely disappeared. Histopathology of the former tumor site would reveal the complete absence of tumor cells. Implantation of additional tumor cells into another region in the back of the cured mouse will result in the rejection of the new tumor cells and prevent the outgrowth of a new tumor, suggesting that the Hsp ProteoCage preparation produced long term memory capable of preventing recurrences. A similar result is expected when the antigen is covalently attached to the Hsp ProteoCage.

### Example 3

Human papilloma Virus Type 18 (HPV 18) is associated with and is considered high risk to cause cervical dysplasia and cervical cancer in women and anal dysplasia and anal cancer in women and men. The E7 protein from HPV 18 is an oncogene which is known to be a transforming agent and is known to be expressed in precancerous and cancerous lesions caused by its continual presence. Once appropriately stimulated, the immune system of the human with such virally induced lesions should be able to eradicate cells expressing antigenic peptides derived from this protein.

To construct a therapeutic vaccine, one that will activate and stimulate the immune system to attack and destroy already existing lesions, the HPV 18 E7 is entrapped in a ProteoCage composed of the small heat shock protein of M. jannaschii. The E7 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT. A similar result is expected when the antigen is covalently attached to the Hsp ProteoCage.

### Example 4

Human papilloma Virus Type 18 (HPV 18) is associated with and is considered high risk to cause cervical dysplasia and cervical cancer in women and anal dysplasia and anal cancer in women and men. The E6 protein from HPV 18 is an oncogene which is known to be a transforming agent and is known to be expressed in precancerous and cancerous lesions caused by its continual presence. Once appropriately stimulated, the immune system of the human with such virally induced lesions should be able to eradicate cells expressing antigenic peptides derived from this protein.

To construct a therapeutic vaccine, one that will activate and stimulate the immune system to attack and destroy already existing lesions, the HPV 18 E6 is entrapped in a Hsp ProteoCage composed of the small heat shock protein of M. jannaschii. The E6 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT. A similar result is expected when the antigen is covalently attached to the Hsp ProteoCage.

### Example 5

Human papilloma Virus Type 6 (HPV 6) is associated with genital warts (condylomata) in women and men. The E7 protein from HPV 6 is an oncogene which is known to be a transforming agent and appears to be present in such lesions. Once appropriately stimulated, the immune system of the human with such virally induced lesions should be able to eradicate cells expressing antigenic peptides derived from this protein.

To construct a therapeutic vaccine, one that will activate and stimulate the immune system to attack and destroy already existing lesions, the HPV 6 E7 is entrapped in a Hsp ProteoCage composed of the small heat shock protein of M. jannaschii. The E7 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells will respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT. A similar result is expected when the antigen is covalently attached to the Hsp ProteoCage.

### Example 6

Human papilloma Virus Type 6 (HPV6) is associated with genital warts (condylomata) in women and men. The E6 protein from HPV 6 is an oncogene which is known to be a transforming agent and appears to be present in such lesions. Once appropriately stimulated, the immune system of the human with such virally induced lesions should be able to eradicate cells expressing antigenic peptides derived from this protein.

To construct a therapeutic vaccine, one that will activate and stimulate the immune system to attack and destroy already existing lesions, the HPV 6 E6 is entrapped in a ProteoCage composed of the small heat shock protein of M. jannaschii. The E6 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells will respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT. A similar result is expected when the antigen is covalently attached to the Hsp ProteoCage.

### Example 7

Human papilloma Virus Type 11 (HPV 11) is associated with genital warts (condylomata) in women and men. The E7 protein from HPV 11 is an oncogene which is known to be a transforming agent and appears to be present in such lesions. Once appropriately stimulated, the immune system of the human with such virally induced lesions should be able to eradicate cells expressing antigenic peptides derived from this protein.

To construct a therapeutic vaccine, one that will activate and stimulate the immune system to attack and destroy already existing lesions, the HPV 11 E7 is entrapped in a ProteoCage composed of the small heat shock protein of M. jannaschii. The E7 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells will respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT. A similar result is expected when the antigen is covalently attached to the Hsp ProteoCage.

### Example 8

Human papilloma Virus Type 11 (HPV11) is associated with genital warts (condylomata) in women and men. The E6 protein from HPV 11 is an oncogene which is known to be a transforming agent and appears to be present in such lesions. Once appropriately stimulated, the immune system of the human with such virally induced lesions should be able to eradicate cells expressing antigenic peptides derived from this protein.

To construct a therapeutic vaccine, one that will activate and stimulate the immune system to attack and destroy already existing lesions, the HPV 11 E6 is entrapped in a Hsp ProteoCage composed of the small heat shock protein of M. jannaschii. The E6 protein or a portion thereof is covalently attached to the inside (or outside) of the Hsp ProteoCage via a suitable linker.

To demonstrate the appropriate activity in vitro, the Hsp ProteoCage containing the viral antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells will respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT. A similar result is expected when the antigen is covalently attached to the Hsp ProteoCage.

### Example 9

Many cancer cells express unique antigens (tumor associated antigens) on their surface. These antigens are rarely or never expressed on normal cells. Therefore, once appropriately stimulated, the immune system of the human with such tumors should be able to eradicate cells expressing antigenic peptides derived from the expressed tumor antigen. For example, the Muc1 antigen is associated with ovarian and breast cancer.

To construct a therapeutic vaccine, one that will activate and stimulate the immune system to attack and destroy already existing lesions, the Muc 1 antigen is entrapped in a Hsp ProteoCage composed of the small heat shock protein of M. jannaschii. The Muc 1 protein or a portion thereof is covalently attached to the inside (or outside) of the Hsp ProteoCage via a suitable linker.

To demonstrate the appropriate activity in vitro, the Hsp ProteoCage containing the cancer antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT.

In order to demonstrate the desired antitumor activity in vivo, a mouse is injected with cancer cells that contain the Muc I antigen and express peptides from that antigen on their surfaces in the context of the cellular MHC I. When the cells have grown to a sufficient size for the tumor to be measurable, the Hsp ProteoCage containing the Muc 1 antigen is injected subcutaneously into the mouse at a site distal to the tumor. The presence or absence of the tumor is easily measured by palpation and the size of the tumor is measured using appropriately sized calipers. When microgram quantities of the Hsp ProteoCage containing Muc 1 are injected subcutaneously under the scruff of the neck of a mouse that has a measurable tumor growing in the distal back of the animal, the tumor will begin to shrink. After 14 to 21 days, the tumor will have completely disappeared. Histopathology of the former tumor site will reveal the complete absence of tumor cells. Implantation of additional tumor cells into another region in the back of the cured mouse would result in the rejection of the new tumor cells and prevent the outgrowth of a new tumor, suggesting that the Hsp ProteoCage preparation produced long term memory capable of preventing recurrences. A similar result is expected when the antigen is covalently attached to the Hsp ProteoCage.

### Example 10

Many cancer cells express unique antigens (tumor associated antigens) on their surface. These antigens are rarely or never expressed on normal cells. Therefore, once appropriately stimulated, the immune system of the human with such tumors should be able to eradicate cells expressing antigenic peptides derived from the expressed tumor antigen. For example, the prostate-specific membrane specific antigen (PSMA) is associated with prostate cancer.

To construct a therapeutic vaccine, one that will activate and stimulate the immune system to attack and destroy already existing lesions, the PSMA antigen is entrapped in a Hsp ProteoCage composed of the small heat shock protein of M. jannaschii. The PMSA protein or a portion thereof is covalently attached to the inside (or outside) of the Hsp ProteoCage via a suitable linker.

To demonstrate the appropriate activity in vitro, the Hsp ProteoCage containing the cancer antigen is dissolved in isotonic buffer and the solution placed into a well in a ninety-six well tissue culture plate in which professional antigen presenting cells reside. After a set period of time, cloned T cells which recognize portions of the antigen in the context of the Major Histocompatibility Complex I (MHC I) are also placed in the wells. Over the next several hours, the T cells will respond by increasing in number and by acquiring antigen specificity (the ability to kill cells expressing the antigen) and by producing cytokines such as Interferon-Gamma (IFNγ). The ability of the cells to kill appropriate target cells is measured in standard Cytotoxic T Cell (CTL) assays well known in the art. The ability to produce IFNγ is measured using methods well known in the art such as Fluorescence Activated Cell Sorting or ELISPOT.

In order to demonstrate the desired antitumor activity in vivo, a mouse is injected with cancer cells that contain the PSMA antigen and express peptides from that antigen on their surfaces in the context of the cellular MUC I. When the cells have grown to a sufficient size for the tumor to be measurable, the Hsp ProteoCage containing the PSMA antigen is injected subcutaneously into the mouse at a site distal to the tumor. The presence or absence of the tumor is easily measured by palpation and the size of the tumor is measured using appropriately sized calipers. When microgram quantities of the Hsp ProteoCage containing PMSA are injected subcutaneously under the scruff of the neck of a mouse that has a measurable tumor growing in the distal back of the animal, the tumor will begin to shrink. After 14 to 21 days, the tumor will have completely disappeared. Histopathology of former tumor site would reveal the complete absence of tumor cells. Implantation of additional tumor cells into another region in the back of the cured mouse will result in the rejection of the new tumor cells and prevent the outgrowth of a new tumor, suggesting that the Hsp ProteoCage preparation produced long term memory capable of preventing recurrences. A similar result is expected when the antigen is covalently attached to the Hsp ProteoCage.

### Example 11

The potency of the preparations used above can be increased by the addition of certain adjuvants such as unmethylated polynucleotide sequences such as CpG's and their derivatives, or polynucleotides such as double stranded poly IC.

When the experiments using the Hsp ProteoCages described in the above examples are performed, a dose response can be recorded as a measure of their activity. An adjuvant, including ones such as unmethylated polynucleotide sequences such as CpG's and their derivatives, or polynucleotides such as double stranded poly IC, may also be entrapped or covalently attached within the Hsp ProteoCage to increase the observed potency.

Alternatively, the adjuvant may be on the outside of the Hsp ProteoCage. The adjuvant may be covalently attached or the Hsp ProteoCage may be formulated in a solution or other preparation in which the adjuvant is dissolved or suspended, and in which the Hsp ProteoCage is also dissolved or suspended.

A murine animal model is employed to demonstrate the appropriate antiviral and antitumor activity in vivo. The TC-1 cell line was created from epithelial cells transformed with HPV E6 and E7 oncogenes and also activated h-RAS. The cell line expresses antigens derived from these proteins. When injected subcutaneously in the distal back of a mouse, the cells grow to produce a nonmetastatic tumor that, when left untreated, overwhelms the mouse, killing it. The presence or absence of the tumor is easily measured by palpation and the size of the tumor is measured using appropriately sized calipers. When microgram quantities of the Hsp ProteoCage containing both the HPV 16 E7 and poly IC or CpG inside the Hsp ProteoCage are injected subcutaneously under the scruff of the neck of a mouse that has a measurable tumor growing in the distal back of the animal, the tumor will begin to shrink. After 14 to 21 days, the tumor will have completely disappeared. Histopathology of the former tumor site would reveal the complete absence of tumor cells. Implantation of additional tumor cells into another region in the back of the cured mouse will result in the rejection of the new tumor cells and prevent the outgrowth of a new tumor, suggesting that the Hsp ProteoCage preparation produced long term memory capable of preventing recurrences. The quantity of Hsp ProteoCages necessary to get this result would be significantly less (10 to 100 fold) than the dose of Hsp ProteoCages without incorporated Poly IC (Example 1). A similar results is expected when the antigen or Poly IC or CpG is covalently attached to the Hsp ProteoCage.

### Example 12

Alternatively, the ProteoCage may be formulated in a solution or other preparation in which the adjuvant is dissolved or suspended in the solution in which the Hsp ProteoCage is also dissolved or suspended.

A murine animal model is employed to demonstrate the appropriate antiviral and antitumor activity in vivo. The TC-1 cell line was created from epithelial cells transformed with HPV E6 and E7 oncogenes and also activated h-RAS. The cell line expresses antigens derived from these proteins. When injected subcutaneously in the distal back of a mouse, the cells grow to produce a nonmetastatic tumor that, when left untreated, overwhelms the mouse, killing it. The presence or absence of the tumor is easily measured by palpation and the size of the tumor is measured using appropriately sized calipers. Microgram quantities of the Hsp ProteoCage containing the HPV 16 E7 inside the Hsp ProteoCage are suspended in a solution containing nanogram to microgram quantities of CpG's or their derivatives (see, e.g., Singh et al., Pharm Res., 19:715-28 (2002); O'Hagan et al., Biomol. Eng., 18:69-85 (2001)). This formulation is injected subcutaneously under the scruff of the neck of a mouse that has a measurable tumor growing in the distal back of the animal, the tumor will begin to shrink. After 14 to 21 days, the tumor will have completely disappeared. Histopathology of the former tumor site reveals the complete absence of tumor cells. Implantation of additional tumor cells into another region in the back of the cured mouse would result in the rejection of the new tumor cells and prevent the outgrowth of a new tumor, suggesting that the Hsp ProteoCage preparation produced long term memory capable of preventing recurrences. The quantity of Hsp ProteoCages necessary to get this result is significantly less (10 to 100 fold) than the dose of Hsp ProteoCages without the external CpG. A similar results is expected if the Hsp 16 E7 antigen is entrapped or covalently attached to the inside or outside of the Hsp ProteoCage.

### Example 13

Once a month for three months, approximately 500 micrograms (1 μg to 2000 μg) of Hsp ProteoCages containing the HPV 16 E7 are injected subcutaneously into a patient with who has been diagnosed with cervical dysplasia. Four to six months after the beginning of the treatment, the patient will undergo a cervical biopsy to determine by standard histopathological procedures that the dysplasia has indeed regressed or been completely eliminated.

Additional courses of therapy may be given as deemed appropriate by the treating physician.

When using a Hsp ProteoCage formulation containing an adjuvant such as Poly IC or a CpG or its derivatives, each dose would be appropriately decreased to match the increase in potency afforded by the presence of the adjuvant.

### Example 14

After appropriate surgical and/or radiation therapy, once a month for three months, approximately 500 micrograms (1 μg to 2000 μg) of Hsp ProteoCages containing the HPV 16 E7 are injected subcutaneously into a patient with who has been diagnosed with cervical cancer. Four to six months after the beginning of the treatment, the patient will undergo a cervical biopsy to determine by standard histopathological procedures that the residual cancer has indeed regressed or been completely eliminated.

Additional courses of therapy may be given as deemed appropriate by the treating physician.

When using a Hsp ProteoCage formulation containing and adjuvant such as Poly IC or a CpG or its derivatives, each dose would be appropriately decreased to match the increase in potency afforded by the presence of the adjuvant.

### Example 15

Once a month for three months, approximately 500 micrograms (1 μg to 2000 μg) of Hsp ProteoCages containing Muc 1 are injected subcutaneously into a patient with who has been diagnosed with ovarian or breast cancer. Four to six months after the beginning of the treatment, the patient will undergo biopsies and/or whole body radiological scans to determine that the previously observed cancer sites have indeed regressed or been completely eliminated.

Additional courses of therapy may be given as deemed appropriate by the treating physician.

When using a Hsp ProteoCage formulation containing and adjuvant such as Poly IC or a CpG or its derivatives, each dose would be appropriately decreased to match the increase in potency afforded by the presence of the adjuvant.

### Example 16

Once a month for three months, approximately 500 micrograms (1 μg to 2000 μg) of Hsp ProteoCages containing PSMA are injected subcutaneously into a patient with who has been diagnosed with cervical dysplasia. Four to six months after the beginning of the treatment, the patient will undergo biopsies and whole body radiological scans to determine that the previously observed cancer sites have indeed regressed or been completely eliminated.

Additional courses of therapy may be given as deemed appropriate by the treating physician.

When using a Hsp ProteoCage formulation containing and adjuvant such as Poly IC or a CpG or its derivatives, each dose would be appropriately decreased to match the increase in potency afforded by the presence of the adjuvant.

### Example 17

The Human Immunodeficiency Virus (HIV) is the cause of immune suppression and its consequences in Acquired Immuno-Deficiency (AIDS). There are several subunits of HIV that would be good targets to which to direct the immune system to destroy cells harboring the virus even when significant viral replication is suppressed by adequate antiretroviral therapy. In the absence of eliminating this reservoir of virus, the potential for return of full-blown disease is never fully eliminated. A therapeutic vaccine directed towards eliminating cells containing virus could significantly advance treatment of patients with HIV.

A Hsp ProteoCage containing an HIV subunit antigen such as gp120 or reverse transcriptase or any other virally encoded protein would induce cellular immunity against cells carrying the virus. Such a Hsp ProteoCage could be constructed with the antigen entrapped or covalently attached as described above. In addition, the Hsp ProteoCage could contain or be formulated with an adjuvant as described above.

Once a month for three months, approximately 500 micrograms (1 μg to 2000 μg) of Hsp ProteoCages containing an appropriate HIV antigen are injected subcutaneously into a patient with who has been diagnosed with the viral infection. At monthly intervals thereafter, the patient undergoes tests to determine if his or her viral titer has decreased and that the need for antiretroviral therapy is decreased.

Additional courses of therapy may be given as deemed appropriate by the treating physician.

When using a ProteoCage formulation containing and adjuvant such as Poly IC or a CpG or its derivatives, each dose would be appropriately decreased to match the increase in potency afforded by the presence of the adjuvant.

### Example 18

Influenza is a virally disease that attacks the general population at regular (seasonal or annual) intervals. The virus undergoes mutations on a regular basis since its antigens face immunological pressure from the affected populations' immunological systems. The mutations usually occur on surface antigens to which humoral (antibody) immunity is established and to which prophylactic vaccines are made. However, there are certain subunits of the virus that do not have such immunological pressure and are relatively constant across all and humans. One of these is the Nuclear Protein (NP).

An Hsp ProteoCage containing an influenza subunit antigen such as NP or any other virally encoded protein would induce cellular immunity against cells carrying the virus. Such a Hsp ProteoCage could be constructed with the antigen entrapped or covalently attached as described above. In addition, the Hsp ProteoCage could contain or be formulated with an adjuvant as described above. Induction of cellular immunity by this construct would be therapeutic in that it could induce cellular immunity that would rid the affected individual of reservoirs that continue to produce the virus, thus helping to shorten the disease symptoms and in fact helping to cure already infected individuals from succumbing to the disease, a problem especially observed in the elderly and those with chronic pulmonary diseases.

Approximately 500 micrograms (1 μg to 2000 μg) of Hsp ProteoCages containing an appropriate influenza antigen are injected subcutaneously into a patient with who has been diagnosed with the viral infection. At appropriate intervals thereafter, the patient undergoes tests to determine his symptoms have declined and the need for supportive therapy and other medications has decreased.

Additional courses of therapy may be given as deemed appropriate by the treating physician.

When using a Hsp ProteoCage formulation containing and adjuvant such as Poly IC or a CpG or its derivatives, each dose would be appropriately decreased to match the increase in potency afforded by the presence of the adjuvant.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A therapeutic vaccine, said therapeutic vaccine comprising:
a heat shock protein assembled into a protein cage, said protein cage having an interior and exterior; and
an antigen.

2. The therapeutic vaccine of claim 1, wherein said heat shock protein is derived from a member selected from the group consisting of a bacterium, a mycobacterium, a yeast, a plant, and an animal.

3. The therapeutic vaccine of claim 1, wherein said heat shock protein is selected from the group consisting of Methannococcus jannaschii Hsp 16.5, Mycobacterium tuberculosis Acr1, Thermococcus sp. strain KS-1 sHsp, Sulfolobus tokodaii strain 7 StHsp 19.7, and Sulfolobus tokodaii strain 7 StHsp 14.0.

4. The therapeutic vaccine of claim 1, wherein the heat shock protein is selected from the group consisting of wheat Hsp 16.9, Saccharomyces pompi spHsp 16.0, Saccharomyces cerevisiae Hsp27, and human Hsp27.

5. The therapeutic vaccine of claim 1, wherein said antigen is within the interior of said protein cage.

6. The therapeutic vaccine of claim 1, wherein said antigen is on the exterior of said protein cage.

7. The therapeutic vaccine of claim 1, wherein said antigen is a member selected from the group consisting of a bacterial antigen, mycobacterial antigen, viral antigen, and tumor antigen.

8. The therapeutic vaccine of claim 7, wherein said tumor antigen is expressed on a cancer cell selected from the group consisting of a melanoma cell, a lymphoma cell, a Hodgkin's Disease cell, an anaplastic large cell cancer, a prostate cancer cell, a Burkitt's lymphoma cell, and a cervical carcinoma cell.

9. The therapeutic vaccine of claim 7, wherein said tumor antigen is a member selected from the group consisting of tumor antigens listed in Tables 1-7.

10. The therapeutic vaccine of claim 7, wherein said viral antigen is derived from a virus selected from the group consisting of herpes simplex virus (HSV), hepatitis B virus (HBV), hepatitis C virus (HCV), cytomegalovirus (CMV), Epstein-Barr virus (EBV), influenza virus, measles virus, human immunodeficiency virus (HIV), and human papilloma virus (HPV).

11. The therapeutic vaccine of claim 1, wherein said antigen is an allergy antigen selected from the group consisting of ragweed, grass, tree pollen, animal dander, and molds.

12. The therapeutic vaccine of claim 1, wherein said therapeutic vaccine further comprises an adjuvant.

13. The therapeutic vaccine of claim 1, wherein said therapeutic vaccine is formulated in an admixture comprising an adjuvant.

14. A method of providing a therapeutic vaccine for the treatment of a disease in a subject, said method comprising administering to said subject a therapeutically effective amount of a therapeutic vaccine comprising a heat shock protein assembled into a protein cage and an antigen, thereby providing treatment to the subject.

15. The method of claim 14, wherein said therapeutic vaccine induces cellular immunity.

16. The method of claim 14, wherein said disease is selected from the group consisting of a bacterial or fungal infectious disease, an acute or chronic viral infection, allergy, and cancer.

17. The method of claim 14, wherein said antigen is a cancer antigen.

18. The method of claim 14, wherein said therapeutic vaccine further comprises an adjuvant.

19. The method of claim 18, wherein said adjuvant is a molecule derived from a member selected from the group consisting of lipid A, muramyl di-peptide (MDP), CpG motifs, or polyI/polyC, endotoxin, and lipopolysaccharide (LPS).

20. The method of claim 14, further comprising the step of administering immunotherapy.


SEQUENCE LISTING (250 sequences)

SEQ ID NO: 1 - protein, 9 aa, Artificial Sequence
  Class I HLA-restricted cancer-testis antigen BAGE peptide epitope
AARAVFLAL

SEQ ID NO: 2 - protein, 11 aa, Artificial Sequence
  Class I HLA-restricted cancer-testis antigen CAMELpeptide epitope
MLMAQEALAFL

SEQ ID NO: 3 - protein, 9 aa, Artificial Sequence
  Class I HLA-restricted cancer-testis antigens DAM-6 and DAM-10 (MAGE-B1 and MAGE-B2) peptide epitope
FLWGPRAYA

SEQ ID NO: 4 - protein, 8 aa, Artificial Sequence
  Class I HLA-restricted cancer-testis antigens GAGE-1, GAGE-2 and GAGE-8 peptide epitope
YRPRPRRY

SEQ ID NO: 5 - protein, 9 aa, Artificial Sequence
  Class I HLA-restricted cancer-testis antigens GAGE-3, GAGE-4, GAGE-5, GAGE-6 and GAGE-7B peptide epitope
YYWPRPRRY

SEQ ID NO: 6 - protein, 9 aa, Artificial Sequence
  Class I HLA-restricted cancer-testis antigen IL-13Ralpha2 peptide epitope
WLPFGFILI

SEQ ID NO: 7 - protein, 9 aa, Artificial Sequence
  Class I HLA-restricted cancer-testis antigen MAGE-A1 peptide epitope
EADPTGHSY

SEQ ID NO: 8 - protein, 9 aa, Artificial Sequence
  Class I HLA-restricted cancer-testis antigen MAGE-A1 peptide epitope
SLFRAVITK

SEQ ID NO: 9 - protein, 9 aa, Artificial Sequence
  Class I HLA-restricted cancer-testis antigen MAGE-A1 peptide epitope
NYKHCFPEI

SEQ ID NO: 10 - protein, 10 aa, Artificial Sequence
  Class I HLA-restricted cancer-testis antigen MAGE-A1 peptide epitope
EVYDGREHSA

SEQ ID NO: 11 - protein, 10 aa, Artificial Sequence
  Class I HLA-restricted cancer-testis antigen MAGE-A1 peptide epitope
REPVTKAEML

SEQ ID NO: 12 - protein, 9 aa, Artificial Sequence
  Class I HLA-restricted cancer-testis antigen MAGE-A1 peptide epitope
DPARYEFLW

SEQ ID NO: 13 - protein, 9 aa, Artificial Sequence
  Class I HLA-restricted cancer-testis antigen MAGE-A1 peptide epitope
SAFPTTINF

SEQ ID NO: 14 - protein, 9 aa, Artificial Sequence
  Class I HLA-restricted cancer-testis antigen MAGE-A1 peptide epitope
SAYGEPRKL

SEQ ID NO: 15 - protein, 9 aa, Artificial Sequence
  Class I HLA-restricted cancer-testis antigen MAGE-A2 peptide epitope
KMVELVHFL

SEQ ID NO: 16 - protein, 10 aa, Artificial Sequence
  Class I HLA-restricted cancer-testis antigen MAGE-A2 peptide epitope
YLQLVFGIEV

SEQ ID NO: 17 - protein, 9 aa, Artificial Sequence
  Class I HLA-restricted cancer-testis antigen MAGE-A2 peptide epitope
EYLQLVFGI

SEQ ID NO: 18 - protein, 10 aa, Artificial Sequence
  Class I HLA-restricted cancer-testis antigens MAGE-A2, MAGE-A3 and MAGE-A6 peptide epitope
REPVTKAEML

SEQ ID NO: 19 - protein, 9 aa, Artificial Sequence
  Class I HLA-restricted cancer-testis antigen MAGE-A3 peptide epitope
EADPIGHLY

SEQ ID NO: 20 - protein, 9 aa, Artificial Sequence
  Class I HLA-restricted cancer-testis antigen MAGE-A3 peptide epitope
FLWGPRALV

SEQ ID NO: 21 - protein, 9 aa, Artificial Sequence
  Class I HLA-restricted cancer-testis antigen MAGE-A3 peptide epitope
TFPDLESEF

SEQ ID NO: 22 - protein, 9 aa, Artificial Sequence
  Class I HLA-restricted cancer-testis antigen MAGE-A3 peptide epitope
IMPKAGLLI

SEQ ID NO: 23 - protein, 10 aa, Artificial Sequence
  Class I HLA-restricted cancer-testis antigen MAGE-A3 peptide epitope
MEVDPIGHLY

SEQ ID NO: 24 - protein, 9 aa, Artificial Sequence
  Class I HLA-restricted cancer-testis antigen MAGE-A3 peptide epitope
WQYFFPVIF

SEQ ID NO: 25 - protein, 9 aa, Artificial Sequence
  Class I HLA-restricted cancer-testis antigen MAGE-A3 peptide epitope
EVDPIGHLY

SEQ ID NO: 26 - protein, 10 aa, Artificial Sequence
  Class I HLA-restricted cancer-testis antigen MAGE-A4 peptide epitope
GVYDGREHTV

SEQ ID NO: 27 - protein, 9 aa, Artificial Sequence
  Class I HLA-restricted cancer-testis antigen MAGE-A6 peptide epitope
MVKISGGPR

SEQ ID NO: 28 - protein, 9 aa, Artificial Sequence
  Class I HLA-restricted cancer-testis antigen MAGE-A6 peptide epitope
EVDPIGHVY

SEQ ID NO: 29 - protein, 9 aa, Artificial Sequence
  Class I HLA-restricted cancer-testis antigen MAGE-A10 peptide epitope
GLYDGMEHL

SEQ ID NO: 30 - protein, 9 aa, Artificial Sequence
  Class I HLA-restricted cancer-testis antigen MAGE-A12 peptide epitope
VRIGHLYIL

SEQ ID NO: 31 - protein, 11 aa, Artificial Sequence
  Class I HLA-restricted cancer-testis antigen NA88-A peptide epitope
MTQGQHFLQKV

SEQ ID NO: 32 - protein, 11 aa, Artificial Sequence
  Class I HLA-restricted cancer-testis antigen NY-ESO-1 peptide epitope
SLLMWITQCFL

SEQ ID NO: 33 - protein, 9 aa, Artificial Sequence
  Class I HLA-restricted cancer-testis antigen NY-ESO-1 peptide epitope
SLLMWITQC

SEQ ID NO: 34 - protein, 9 aa, Artificial Sequence
  Class I HLA-restricted cancer-testis antigen NY-ESO-1 peptide epitope
QLSLLMWIT

SEQ ID NO: 35 - protein, 9 aa, Artificial Sequence
  Class I HLA-restricted cancer-testis antigen NY-ESO-1 peptide epitope
MPFATPMEA

SEQ ID NO: 36 - protein, 10 aa, Artificial Sequence
  Class I HLA-restricted cancer-testis antigen NY-ESO-1a (CAG-3) peptide epitope
ASGPGGGAPR

SEQ ID NO: 37 - protein, 9 aa, Artificial Sequence
  Class I HLA-restricted cancer-testis antigen SSX-2peptide epitope
KASEKIFYV

SEQ ID NO: 38 - protein, 9 aa, Artificial Sequence
  Class I HLA-restricted cancer-testis antigen TRAG-3 peptide epitope
ILLRDAGLV

SEQ ID NO: 39 - protein, 9 aa, Artificial Sequence
  Class I HLA-restricted differentiation antigen CEA (CAP-1) peptide epitope
YLSGANLNL

SEQ ID NO: 40 - protein, 9 aa, Artificial Sequence
  Class I HLA-restricted cancer-testis antigen CEA peptide epitope
HLFGYSWYK

SEQ ID NO: 41 - protein, 9 aa, Artificial Sequence
  Class I HLA-restricted differentiation antigen Ep-CAM peptide epitope
GLKAGVIAV

SEQ ID NO: 42 - protein, 9 aa, Artificial Sequence
  Class I HLA-restricted differentiation antigen Gp100 peptide epitope
KTWGQYWQV

SEQ ID NO: 43 - protein, 10 aa, Artificial Sequence
  Class I HLA-restricted differentiation antigen Gp100 peptide epitope
AMLGTHTMEV

SEQ ID NO: 44 - protein, 9 aa, Artificial Sequence
  Class I HLA-restricted differentiation antigen Gp100 peptide epitope
MLGTHTMEV

SEQ ID NO: 45 - protein, 10 aa, Artificial Sequence
  Class I HLA-restricted differentiation antigen Gp100 peptide epitope
SLADTNSLAV

SEQ ID NO: 46 - protein, 9 aa, Artificial Sequence
  Class I HLA-restricted differentiation antigen Gp100 peptide epitope
ITDQVPFSV

SEQ ID NO: 47 - protein, 10 aa, Artificial Sequence
  Class I HLA-restricted differentiation antigen Gp100 peptide epitope
LLDGTATLRL

SEQ ID NO: 48 - protein, 9 aa, Artificial Sequence
  Class I HLA-restricted differentiation antigen Gp100 peptide epitope
YLEPGPVTA

SEQ ID NO: 49 - protein, 10 aa, Artificial Sequence
  Class I HLA-restricted differentiation antigen Gp100 peptide epitope
VLYRYGSFSV

SEQ ID NO: 50 - protein, 9 aa, Artificial Sequence
  Class I HLA-restricted differentiation antigen Gp100 peptide epitope
RLMKQDFSV

SEQ ID NO: 51 - protein, 9 aa, Artificial Sequence
  Class I HLA-restricted differentiation antigen Gp100 peptide epitope
RLPRIFCSC

SEQ ID NO: 52 - protein, 9 aa, Artificial Sequence
  Class I HLA-restricted differentiation antigen Gp100 peptide epitope
LIYRRRLMK

SEQ ID NO: 53 - protein, 9 aa, Artificial Sequence
  Class I HLA-restricted differentiation antigen Gp100 peptide epitope
ALNFPGSQK

SEQ ID NO: 54 - protein, 10 aa, Artificial Sequence
  Class I HLA-restricted differentiation antigen Gp100 peptide epitope
SLIYRRRLMK

SEQ ID NO: 55 - protein, 9 aa, Artificial Sequence
  Class I HLA-restricted differentiation antigen Gp100 peptide epitope
ALLAVGATK

SEQ ID NO: 56 - protein, 9 aa, Artificial Sequence
  Class I HLA-restricted differentiation antigen Gp100 peptide epitope
VYFFLPDHL

SEQ ID NO: 57 - protein, 10 aa, Artificial Sequence
  Class I HLA-restricted differentiation antigen Gp100 peptide epitope
HTMEVTVYHR

SEQ ID NO: 58 - protein, 10 aa, Artificial Sequence
  Class I HLA-restricted differentiation antigen Gp100 peptide epitope
VPLDCVLYRY

SEQ ID NO: 59 - protein, 8 aa, Artificial Sequence
  Class I HLA-restricted differentiation antigen Gp100 peptide epitope
SNDGPTLI

SEQ ID NO: 60 - protein, 9 aa, Artificial Sequence
  Class I HLA-restricted differentiation antigen mammaglobin-A peptide epitope
PLLENVISK

SEQ ID NO: 61 - protein, 9 aa, Artificial Sequence
  Class I HLA-restricted differentiation antigen mammaglobin-A peptide epitope
KLLMVLMLA

SEQ ID NO: 62 - protein, 9 aa, Artificial Sequence
  Class I HLA-restricted differentiation antigen mammaglobin-A peptide epitope
TTNAIDELK

SEQ ID NO: 63 - protein, 9 aa, Artificial Sequence
  Class I HLA-restricted differentiation antigen mammaglobin-A peptide epitope
AIDELKECF

SEQ ID NO: 64 - protein, 9 aa, Artificial Sequence
  Class I HLA-restricted differentiation antigen melan-A/MART-1 peptide epitope
AAGIGILTV

SEQ ID NO: 65 - protein, 10 aa, Artificial Sequence
  Class I HLA-restricted differentiation antigen melan-A/MART-1 peptide epitope
EAAGIGILTV

SEQ ID NO: 66 - protein, 9 aa, Artificial Sequence
  Class I HLA-restricted differentiation antigen melan-A/MART-1 peptide epitope
ILTVILGVL

SEQ ID NO: 67 - protein, 10 aa, Artificial Sequence
  Class I HLA-restricted differentiation antigen melan-A/MART-1 peptide epitope
AEEAAGIGIL

SEQ ID NO: 68 - protein, 11 aa, Artificial Sequence
  Class I HLA-restricted differentiation antigen melan-A/MART-1 peptide epitope
AEEAAGIGILT

SEQ ID NO: 69 - protein, 9 aa, Artificial Sequence
  Class I HLA-restricted differentiation antigen MC1R peptide epitope
TILLGIFFL

SEQ ID NO: 70 - protein, 9 aa, Artificial Sequence
  Class I HLA-restricted differentiation antigen MC1R peptide epitope
FLALIICNA

SEQ ID NO: 71 - protein, 9 aa, Artificial Sequence
  Class I HLA-restricted differentiation antigen OA1 peptide epitope
LYSACFWWL

SEQ ID NO: 72 - protein, 9 aa, Artificial Sequence
  Class I HLA-restricted differentiation antigen Ppolypeptide peptide epitope
IMLCLIAAV

SEQ ID NO: 73 - protein, 10 aa, Artificial Sequence
  Class I HLA-restricted differentiation antigen PSA peptide epitope
VSHSFPHPLY

SEQ ID NO: 74 - protein, 10 aa, Artificial Sequence
  Class I HLA-restricted differentiation antigen PSA peptide epitope
FLTPKKLQCV

SEQ ID NO: 75 - protein, 10 aa, Artificial Sequence
  Class I HLA-restricted differentiation antigen PSA peptide epitope
VISNDVCAQV

SEQ ID NO: 76 - protein, 9 aa, Artificial Sequence
  Class I HLA-restricted differentiation antigen TRP-1 (gp75) peptide epitope
MSLQRQFLR

SEQ ID NO: 77 - protein, 10 aa, Artificial Sequence
  Class I HLA-restricted differentiation antigen TRP-2 peptide epitope
SVYDFFVWLC

SEQ ID NO: 78 - protein, 9 aa, Artificial Sequence
  Class I HLA-restricted differentiation antigen TRP-2 peptide epitope
TLDSQVMSL

SEQ ID NO: 79 - protein, 9 aa, Artificial Sequence
  Class I HLA-restricted differentiation antigen TRP-2 peptide epitope
LLGPGRPYR

SEQ ID NO: 80 - protein, 9 aa, Artificial Sequence
  Class I HLA-restricted differentiation antigen TRP-2 peptide epitope
ANDPIFVVL

SEQ ID NO: 81 - protein, 9 aa, Artificial Sequence
  Class I HLA-restricted differentiation antigen tyrosinase peptide epitope
KCDICTDEY

SEQ ID NO: 82 - protein, 11 aa, Artificial Sequence
  Class I HLA-restricted differentiation antigen tyrosinase peptide epitope
SSDYVIPIGTY

SEQ ID NO: 83 - protein, 9 aa, Artificial Sequence
  Class I HLA-restricted differentiation antigen tyrosinase peptide epitope
YMDGTMSQV

SEQ ID NO: 84 - protein, 9 aa, Artificial Sequence
  Class I HLA-restricted differentiation antigen tyrosinase peptide epitope
MLLAVLYCL

SEQ ID NO: 85 - protein, 9 aa, Artificial Sequence
  Class I HLA-restricted differentiation antigen tyrosinase peptide epitope
AFLPWHRLF

SEQ ID NO: 86 - protein, 9 aa, Artificial Sequence
  Class I HLA-restricted differentiation antigen tyrosinase peptide epitope
SEIWRDIDF

SEQ ID NO: 87 - protein, 12 aa, Artificial Sequence
  Class I HLA-restricted differentiation antigen tyrosinase peptide epitope
TPRLPSSADVEF

SEQ ID NO: 88 - protein, 9 aa, Artificial Sequence
  Class I HLA-restricted widely occurring, overexpressed antigen adipophilin peptide epitope
SVASTITGV

SEQ ID NO: 89 - protein, 10 aa, Artificial Sequence
  Class I HLA-restricted widely occurring, overexpressed antigen AIM-2 unspliced transcript containing intron 2 peptide epitope
RSDSGQQARY

SEQ ID NO: 90 - protein, 9 aa, Artificial Sequence
  Class I HLA-restricted widely occurring, overexpressed antigen AFP peptide epitope
GVALQTMKQ

SEQ ID NO: 91 - protein, 8 aa, Artificial Sequence
  Class I HLA-restricted widely occurring, overexpressed antigen ART-4 peptide epitope
AFLRHAAL

SEQ ID NO: 92 - protein, 10 aa, Artificial Sequence
  Class I HLA-restricted widely occurring, overexpressed antigen ART-4 peptide epitope
DYPSLSATDI

SEQ ID NO: 93 - protein, 9 aa, Artificial Sequence
  Class I HLA-restricted widely occurring, overexpressed antigen CLCA2 peptide epitope
LLGNCLPTV

SEQ ID NO: 94 - protein, 9 aa, Artificial Sequence
  Class I HLA-restricted widely occurring, overexpressed antigen CLCA2 peptide epitope
SLQALKVTV

SEQ ID NO: 95 - protein, 9 aa, Artificial Sequence
  Class I HLA-restricted widely occurring, overexpressed antigen Cyp-B peptide epitope
KFHRVIKDF

SEQ ID NO: 96 - protein, 9 aa, Artificial Sequence
  Class I HLA-restricted widely occurring, overexpressed antigen Cyp-B peptide epitope
DFMIQGGDF

SEQ ID NO: 97 - protein, 9 aa, Artificial Sequence
  Class I HLA-restricted widely occurring, overexpressed antigen EphA2 peptide epitope
IMNDMPIYM

SEQ ID NO: 98 - protein, 9 aa, Artificial Sequence
  Class I HLA-restricted widely occurring, overexpressed antigen EphA2 peptide epitope
VLAGVGFFI

SEQ ID NO: 99 - protein, 9 aa, Artificial Sequence
  Class I HLA-restricted widely occurring, overexpressed antigen FGF-5 peptide epitope generated by post-translational protein splicing
NTYASPRFK

SEQ ID NO: 100 - protein, 9 aa, Artificial Sequence
  Class I HLA-restricted widely occurring, overexpressed antigen G250 peptide epitope
HLSTAFARV

SEQ ID NO: 101 - protein, 9 aa, Artificial Sequence
  Class I HLA-restricted widely occurring, overexpressed antigen GnT-V peptide epitope within intronic sequence
VLPDVFIRC

SEQ ID NO: 102 - protein, 10 aa, Artificial Sequence
  Class I HLA-restricted widely occurring, overexpressed antigen GnT-V peptide epitope within intronic sequence
VLPDVFIRCV

SEQ ID NO: 103 - protein, 9 aa, Artificial Sequence
  Class I HLA-restricted widely occurring, overexpressed antigen HER-2/neu peptide epitope
KIFGSLAFL

SEQ ID NO: 104 - protein, 9 aa, Artificial Sequence
  Class I HLA-restricted widely occurring, overexpressed antigen HER-2/neu peptide epitope
IISAVVGIL

SEQ ID NO: 105 - protein, 9 aa, Artificial Sequence
  Class I HLA-restricted widely occurring, overexpressed antigen HER-2/neu peptide epitope
RLLQETELV

SEQ ID NO: 106 - protein, 9 aa, Artificial Sequence
  Class I HLA-restricted widely occurring, overexpressed antigen HER-2/neu peptide epitope
VVLGVVFGI

SEQ ID NO: 107 - protein, 9 aa, Artificial Sequence
  Class I HLA-restricted widely occurring, overexpressed antigen HER-2/neu peptide epitope
ILHNGAYSL

SEQ ID NO: 108 - protein, 10 aa, Artificial Sequence
  Class I HLA-restricted widely occurring, overexpressed antigen HER-2/neu peptide epitope
YMIMVKCWMI

SEQ ID NO: 109 - protein, 9 aa, Artificial Sequence
  Class I HLA-restricted widely occurring, overexpressed antigen HER-2/neu peptide epitope
TYLPTNASL

SEQ ID NO: 110 - protein, 9 aa, Artificial Sequence
  Class I HLA-restricted widely occurring, overexpressed antigen HER-2/neu peptide epitope
VLRENTSPK

SEQ ID NO: 111 - protein, 10 aa, Artificial Sequence
  Class I HLA-restricted widely occurring, overexpressed antigen HST-2 (FGF-6) peptide epitope
YSWMDISCWI

SEQ ID NO: 112 - protein, 9 aa, Artificial Sequence
  Class I HLA-restricted widely occurring, overexpressed antigen hTERT peptide epitope
ILAKFLHWL

SEQ ID NO: 113 - protein, 9 aa, Artificial Sequence
  Class I HLA-restricted widely occurring, overexpressed antigen hTERT peptide epitope
RLVDDFLLV

SEQ ID NO: 114 - protein, 9 aa, Artificial Sequence
  Class I HLA-restricted widely occurring, overexpressed antigen hTERT peptide epitope
KLFGVLRLK

SEQ ID NO: 115 - protein, 9 aa, Artificial Sequence
  Class I HLA-restricted widely occurring, overexpressed antigen iCE peptide epitope
SPRWWPTCL

SEQ ID NO: 116 - protein, 9 aa, Artificial Sequence
  Class I HLA-restricted widely occurring, overexpressed antigen livin (ML-IAP) peptide epitope
SLGSPVLGL

SEQ ID NO: 117 - protein, 10 aa, Artificial Sequence
  Class I HLA-restricted widely occurring, overexpressed antigen livin (ML-IAP) peptide epitope
RLASFYDWPL

SEQ ID NO: 118 - protein, 14 aa, Artificial Sequence
  Class I HLA-restricted widely occurring, overexpressed antigen M-CSF alternative ORF peptide epitope
LPAVVGLSPGEQEY

SEQ ID NO: 119 - protein, 9 aa, Artificial Sequence
  Class I HLA-restricted widely occurring, overexpressed antigen MUC1 peptide epitope
STAPPAHGV

SEQ ID NO: 120 - protein, 9 aa, Artificial Sequence
  Class I HLA-restricted widely occurring, overexpressed antigen MUC1 peptide epitope
STAPPVHNV

SEQ ID NO: 121 - protein, 9 aa, Artificial Sequence
  Class I HLA-restricted widely occurring, overexpressed antigen MUC2 peptide epitope
LLNQLQVNL

SEQ ID NO: 122 - protein, 9 aa, Artificial Sequence
  Class I HLA-restricted widely occurring, overexpressed antigen MUC2 peptide epitope
MLWGWREHV

SEQ ID NO: 123 - protein, 9 aa, Artificial Sequence
  Class I HLA-restricted widely occurring, overexpressed antigen PRAME peptide epitope
LYVDSLFFL

SEQ ID NO: 124 - protein, 9 aa, Artificial Sequence
  Class I HLA-restricted widely occurring, overexpressed antigen PRAME peptide epitope
VLDGLDVLL

SEQ ID NO: 125 - protein, 10 aa, Artificial Sequence
  Class I HLA-restricted widely occurring, overexpressed antigen PRAME peptide epitope
SLYSFPEPEA

SEQ ID NO: 126 - protein, 10 aa, Artificial Sequence
  Class I HLA-restricted widely occurring, overexpressed antigen PRAME peptide epitope
ALYVDSLFFL

SEQ ID NO: 127 - protein, 9 aa, Artificial Sequence
  Class I HLA-restricted widely occurring, overexpressed antigen PRAME peptide epitope
SLLQHLIGL

SEQ ID NO: 128 - protein, 10 aa, Artificial Sequence
  Class I HLA-restricted widely occurring, overexpressed antigen PSM A peptide epitope
HSTNGVTRIY

SEQ ID NO: 129 - protein, 9 aa, Artificial Sequence
  Class I HLA-restricted widely occurring, overexpressed antigen PSM A peptide epitope
LYSDPADYF

SEQ ID NO: 130 - protein, 9 aa, Artificial Sequence
  Class I HLA-restricted widely occurring, overexpressed antigen PSM A peptide epitope
NYARTEDFF

SEQ ID NO: 131 - protein, 9 aa, Artificial Sequence
  Class I HLA-restricted widely occurring, overexpressed antigen P15 peptide epitope
AYGLDFYIL

SEQ ID NO: 132 - protein, 9 aa, Artificial Sequence
  Class I HLA-restricted widely occurring, overexpressed antigen P53 peptide epitope
AIYKQSQHM

SEQ ID NO: 133 - protein, 9 aa, Artificial Sequence
  Class I HLA-restricted widely occurring, overexpressed antigen mutated P53 peptide epitope
SQKTYQGSY

SEQ ID NO: 134 - protein, 10 aa, Artificial Sequence
  Class I HLA-restricted widely occurring, overexpressed antigen RAGE peptide epitope
SPSSNRIRNT

SEQ ID NO: 135 - protein, 9 aa, Artificial Sequence
  Class I HLA-restricted widely occurring, overexpressed antigen RU1 peptide epitope
VPYGSFKHV

SEQ ID NO: 136 - protein, 9 aa, Artificial Sequence
  Class I HLA-restricted widely occurring, overexpressed antigen RU2 peptide epitope
LPRWPPPQL

SEQ ID NO: 137 - protein, 9 aa, Artificial Sequence
  Class I HLA-restricted widely occurring, overexpressed antigen SART-1 peptide epitope
EYRGFTQDF

SEQ ID NO: 138 - protein, 9 aa, Artificial Sequence
  Class I HLA-restricted widely occurring, overexpressed antigen SART-1 peptide epitope
KGSGKMKTE

SEQ ID NO: 139 - protein, 9 aa, Artificial Sequence
  Class I HLA-restricted widely occurring, overexpressed antigen SART-2 peptide epitope
DYSARWNEI

SEQ ID NO: 140 - protein, 9 aa, Artificial Sequence
  Class I HLA-restricted widely occurring, overexpressed antigen SART-2 peptide epitope
AYDFLYNYL

SEQ ID NO: 141 - protein, 9 aa, Artificial Sequence
  Class I HLA-restricted widely occurring, overexpressed antigen SART-2 peptide epitope
SYTRLFLIL

SEQ ID NO: 142 - protein, 10 aa, Artificial Sequence
  Class I HLA-restricted widely occurring, overexpressed antigen SART-3 peptide epitope
VYDYNCHVDL

SEQ ID NO: 143 - protein, 9 aa, Artificial Sequence
  Class I HLA-restricted widely occurring, overexpressed antigen SART-3 peptide epitope
AYIDFEMKI

SEQ ID NO: 144 - protein, 9 aa, Artificial Sequence
  Class I HLA-restricted widely occurring, overexpressed antigen SART-3 peptide epitope
LLQAEAPRL

SEQ ID NO: 145 - protein, 9 aa, Artificial Sequence
  Class I HLA-restricted widely occurring, overexpressed antigen SART-3 peptide epitope
RLAEYQAYI

SEQ ID NO: 146 - protein, 10 aa, Artificial Sequence
  Class I HLA-restricted widely occurring, overexpressed antigen SOX10 peptide epitope
SAWISKPPGV

SEQ ID NO: 147 - protein, 10 aa, Artificial Sequence
  Class I HLA-restricted widely occurring, overexpressed antigen survivin peptide epitope
ELTLGEFLKL

SEQ ID NO: 148 - protein, 10 aa, Artificial Sequence
  Class I HLA-restricted widely occurring, overexpressed antigen survivin peptide epitope
TLPPAWQPFL

SEQ ID NO: 149 - protein, 9 aa, Artificial Sequence
  Class I HLA-restricted widely occurring, overexpressed antigen survivin-2B splicing variant peptide epitope
AYACNTSTL

SEQ ID NO: 150 - protein, 9 aa, Artificial Sequence
  Class I HLA-restricted widely occurring, overexpressed antigen TRG in intron of putative tumor suppressor testin peptide epitope
YQLCLTNIF

SEQ ID NO: 151 - protein, 9 aa, Artificial Sequence
  Class I HLA-restricted widely occurring, overexpressed antigen WTI peptide epitope
RMFPNAPYL

SEQ ID NO: 152 - protein, 9 aa, Artificial Sequence
  Class I HLA-restricted widely occurring, overexpressed antigen WTI peptide epitope
CMTWNQMNL

SEQ ID NO: 153 - protein, 9 aa, Artificial Sequence
  Class I HLA-restricted widely occurring, overexpressed antigen WTI peptide epitope
RWPSCQKKF

SEQ ID NO: 154 - protein, 9 aa, Artificial Sequence
  Class I HLA-restricted unidentified antigen 707-AP peptide epitope
RVAALARDA

SEQ ID NO: 155 - protein, 10 aa, Artificial Sequence
  Class I HLA-restricted tumor-specific antigen mutant alpha-actinin-4 peptide epitope
FIASNGVKLV

SEQ ID NO: 156 - protein, 9 aa, Artificial Sequence
  Class I HLA-restricted tumor-specific antigen mutant beta-catenin peptide epitope
SYLDSGIHF

SEQ ID NO: 157 - protein, 9 aa, Artificial Sequence
  Class I HLA-restricted tumor-specific antigen splicing mutant caspase-8 peptide epitope
FPSDSWCYF

SEQ ID NO: 158 - protein, 10 aa, Artificial Sequence
  Class I HLA-restricted tumor-specific antigen mutant CDK-4 peptide epitope
ACDPHSGHFV

SEQ ID NO: 159 - protein, 9 aa, Artificial Sequence
  Class I HLA-restricted tumor-specific antigen mutant ELF2 peptide epitope
ETVSEQSNV

SEQ ID NO: 160 - protein, 13 aa, Artificial Sequence
  Class I HLA-restricted tumor-specific antigen mutant HLA-A*0201-R170I peptide epitope
CVEWLRIYLENGK

SEQ ID NO: 161 - protein, 9 aa, Artificial Sequence
  Class I HLA-restricted tumor-specific antigen mutant HSP70-2 M peptide epitope
SLFEGIDIY

SEQ ID NO: 162 - protein, 9 aa, Artificial Sequence
  Class I HLA-restricted tumor-specific antigen mutant KIAA0205 peptide epitope
AEPINIQTV

SEQ ID NO: 163 - protein, 9 aa, Artificial Sequence
  Class I HLA-restricted tumor-specific antigen mutant malic enzyme peptide epitope
FLDEFMEGV

SEQ ID NO: 164 - protein, 11 aa, Artificial Sequence
  Class I HLA-restricted tumor-specific antigen mutant MART-2 peptide epitope
FLEGNEVGKTY

SEQ ID NO: 165 - protein, 9 aa, Artificial Sequence
  Class I HLA-restricted tumor-specific antigen mutant MUM-1 peptide epitope
EEKLIVVLF

SEQ ID NO: 166 - protein, 10 aa, Artificial Sequence
  Class I HLA-restricted tumor-specific antigen mutant MUM-2 peptide epitope
SELFRSGLDY

SEQ ID NO: 167 - protein, 9 aa, Artificial Sequence
  Class I HLA-restricted tumor-specific antigen mutant MUM-2 peptide epitope
FRSGLDSYV

SEQ ID NO: 168 - protein, 9 aa, Artificial Sequence
  Class I HLA-restricted tumor-specific antigen mutant MUM-3 peptide epitope
EAFIQPITR

SEQ ID NO: 169 - protein, 9 aa, Artificial Sequence
  Class I HLA-restricted tumor-specific antigen mutant myosin peptide epitope
KINKNPKYK

SEQ ID NO: 170 - protein, 9 aa, Artificial Sequence
  Class I HLA-restricted tumor-specific antigen mutant OS-9 peptide epitope
KELEGILLL

SEQ ID NO: 171 - protein, 10 aa, Artificial Sequence
  Class I HLA-restricted tumor-specific antigen BING-4 alternative ORF peptide epitope
MCQWGRLWQL

SEQ ID NO: 172 - protein, 9 aa, Artificial Sequence
  Class I HLA-restricted tumor-specific antigen mutant K-RAS peptide epitope
VVVGAVGVG

SEQ ID NO: 173 - protein, 10 aa, Artificial Sequence
  Class I HLA-restricted tumor-specific antigen mutant N-RAS peptide epitope
ILDTAGREEY

SEQ ID NO: 174 - protein, 10 aa, Artificial Sequence
  Class I HLA-restricted tumor-specific antigen OGT translational frameshift peptide epitope
SLYKFSPFPL

SEQ ID NO: 175 - protein, 10 aa, Artificial Sequence
  Class I HLA-restricted tumor-specific antigen TGFalphaRII translational frameshift  peptide epitope
RLSSCVPVVA

SEQ ID NO: 176 - protein, 10 aa, Artificial Sequence
  Class I HLA-restricted tumor-specific antigen TRP-2/INT2 intronic sequence  peptide epitope
EVISCKLIKR

SEQ ID NO: 177 - protein, 9 aa, Artificial Sequence
  Class I HLA-restricted tumor-specific antigen TRP-2-6b alternatively spliced exon 6b from intron 6 peptide epitope
ATTNILEHY

SEQ ID NO: 178 - protein, 8 aa, Artificial Sequence
  Class II HLA-restricted cancer-testis antigen CAMEL peptide epitope
PWKRSWSA

SEQ ID NO: 179 - protein, 13 aa, Artificial Sequence
  Class II HLA-restricted cancer-testis antigen LAGE-1 peptide epitope
ILSRDAAPLPRPG

SEQ ID NO: 180 - protein, 14 aa, Artificial Sequence
  Class II HLA-restricted cancer-testis antigens MAGE-A1, MAGE-A2, MAGE-A3 and MAGE-A6 peptide epitope
LLKYRAREPVTKAE

SEQ ID NO: 181 - protein, 15 aa, Artificial Sequence
  Class II HLA-restricted cancer-testis antigen MAGE-A3 peptide epitope
TSYVKVLHHMVKISG

SEQ ID NO: 182 - protein, 14 aa, Artificial Sequence
  Class II HLA-restricted cancer-testis antigen MAGE-A3 peptide epitope
AELVHFLLLKYRAR

SEQ ID NO: 183 - protein, 15 aa, Artificial Sequence
  Class II HLA-restricted cancer-testis antigen MAGE-A3 peptide epitope
RKVAELVHFLLLKYR

SEQ ID NO: 184 - protein, 15 aa, Artificial Sequence
  Class II HLA-restricted cancer-testis antigen MAGE-A3 peptide epitope
GDNQIMPKAGLLIIV

SEQ ID NO: 185 - protein, 15 aa, Artificial Sequence
  Class II HLA-restricted cancer-testis antigen MAGE-A3 peptide epitope
FFPVIFSKASSSLQL

SEQ ID NO: 186 - protein, 15 aa, Artificial Sequence
  Class II HLA-restricted cancer-testis antigen MAGE-A6 peptide epitope
ESEFQAALSRKVAKL

SEQ ID NO: 187 - protein, 24 aa, Artificial Sequence
  Class II HLA-restricted cancer-testis antigen MAGE-A6 peptide epitope
LLKYRAREPVTKAEMLGSVVGNWQ

SEQ ID NO: 188 - protein, 31 aa, Artificial Sequence
  Class II HLA-restricted cancer-testis antigen MAGE-A6 peptide epitope
VGNWQYFFPVIFSKASDSLQLVFGIELMEVD

SEQ ID NO: 189 - protein, 16 aa, Artificial Sequence
  Class II HLA-restricted cancer-testis antigen MAGE-A6 peptide epitope
IFSKASDSLQLVFGIE

SEQ ID NO: 190 - protein, 18 aa, Artificial Sequence
  Class II HLA-restricted cancer-testis antigen MAGE-A6 peptide epitope
LTQYFVQENYLEYRQVPG

SEQ ID NO: 191 - protein, 10 aa, Artificial Sequence
  Class II HLA-restricted cancer-testis antigen NY-ESO-1 peptide epitope
VLLKEFTVSG

SEQ ID NO: 192 - protein, 18 aa, Artificial Sequence
  Class II HLA-restricted cancer-testis antigen NY-ESO-1 peptide epitope
PLPVPGVLLKEFTVSGNI

SEQ ID NO: 193 - protein, 18 aa, Artificial Sequence
  Class II HLA-restricted cancer-testis antigen NY-ESO-1 peptide epitope
VLLKEFTVSGNILTIRLT

SEQ ID NO: 194 - protein, 18 aa, Artificial Sequence
  Class II HLA-restricted cancer-testis antigen NY-ESO-1 peptide epitope
AADHRQLQLSISSCLQQL

SEQ ID NO: 195 - protein, 15 aa, Artificial Sequence
  Class II HLA-restricted differentiation antigen CEA peptide epitope
YACFVSNLATGRNNS

SEQ ID NO: 196 - protein, 13 aa, Artificial Sequence
  Class II HLA-restricted differentiation antigen CEA peptide epitope
LWWVNNQSLPVSP

SEQ ID NO: 197 - protein, 16 aa, Artificial Sequence
  Class II HLA-restricted differentiation antigen Gp100 peptide epitope
WNRQLYPEWTEAQRLD

SEQ ID NO: 198 - protein, 17 aa, Artificial Sequence
  Class II HLA-restricted differentiation antigen Gp100 peptide epitope
TGRAMLGTHTMEVTVYH

SEQ ID NO: 199 - protein, 19 aa, Artificial Sequence
  Class II HLA-restricted differentiation antigen Gp100 peptide epitope
IYRRRLMKQDFSVPQLPHS

SEQ ID NO: 200 - protein, 23 aa, Artificial Sequence
  Class II HLA-restricted differentiation antigen MART-1/melan-A peptide epitope
RNGYRALMDKSLHVGTQCALTRR

SEQ ID NO: 201 - protein, 15 aa, Artificial Sequence
  Class II HLA-restricted differentiation antigen PSA peptide epitope
ILLGRMSLFMPEDTG

SEQ ID NO: 202 - protein, 13 aa, Artificial Sequence
  Class II HLA-restricted differentiation antigen PSA peptide epitope
SLFHPEDTGQVFQ

SEQ ID NO: 203 - protein, 15 aa, Artificial Sequence
  Class II HLA-restricted differentiation antigen PSA peptide epitope
QVFQVSHSFPHPLYD

SEQ ID NO: 204 - protein, 15 aa, Artificial Sequence
  Class II HLA-restricted differentiation antigen PSA peptide epitope
NDLMLLRLSEPAELT

SEQ ID NO: 205 - protein, 13 aa, Artificial Sequence
  Class II HLA-restricted differentiation antigen PSA peptide epitope
KKLQCVQLHVISM

SEQ ID NO: 206 - protein, 15 aa, Artificial Sequence
  Class II HLA-restricted differentiation antigen PSA peptide epitope
GVLQGITSMGSEPCA

SEQ ID NO: 207 - protein, 15 aa, Artificial Sequence
  Class II HLA-restricted differentiation antigen tyrosinase peptide epitope
QNILLSNAPLGPQFP

SEQ ID NO: 208 - protein, 15 aa, Artificial Sequence
  Class II HLA-restricted differentiation antigen tyrosinase peptide epitope
DYSYLQDSDPDSFQD

SEQ ID NO: 209 - protein, 13 aa, Artificial Sequence
  Class II HLA-restricted differentiation antigen tyrosinase peptide epitope
SYLQDSDPDSFQD

SEQ ID NO: 210 - protein, 21 aa, Artificial Sequence
  Class II HLA-restricted differentiation antigen tyrosinase peptide epitope
RHRPLQEVYPEANAPIGHNRE

SEQ ID NO: 211 - protein, 11 aa, Artificial Sequence
  Class II HLA-restricted differentiation antigen tyrosinase peptide epitope
EIWRDIDFAHE

SEQ ID NO: 212 - protein, 20 aa, Artificial Sequence
  Class II HLA-restricted differentiation antigen tyrosinase peptide epitope
YGQMKNGSTPMFNDINIYDL

SEQ ID NO: 213 - protein, 17 aa, Artificial Sequence
  Class II HLA-restricted differentiation antigen tyrosinase peptide epitope
ALHIYMDGTMSQVQGSA

SEQ ID NO: 214 - protein, 16 aa, Artificial Sequence
  Class II HLA-restricted widely expressed antigen annexin II peptide epitope
DVPKWISIMTERSVPH

SEQ ID NO: 215 - protein, 12 aa, Artificial Sequence
  Class II HLA-restricted widely expressed antigen EphA3 peptide epitope
DVTFNIICKKCG

SEQ ID NO: 216 - protein, 12 aa, Artificial Sequence
  Class II HLA-restricted widely expressed antigen HER-2/neu peptide epitope
GSYVSRLLGICL

SEQ ID NO: 217 - protein, 16 aa, Artificial Sequence
  Class II HLA-restricted widely expressed antigen HER-2/neu peptide epitope
VPIKWMALESILRRRF

SEQ ID NO: 218 - protein, 12 aa, Artificial Sequence
  Class II HLA-restricted widely expressed antigen MUC1 peptide epitope
PGSTAPPAHGVT

SEQ ID NO: 219 - protein, 15 aa, Artificial Sequence
  Class II HLA-restricted widely expressed antigen WT1 peptide epitope
PQQMGSDVRDLNALL

SEQ ID NO: 220 - protein, 12 aa, Artificial Sequence
  Class II HLA-restricted mutated CDC27 peptide epitope
FSWAMDLDPKGA

SEQ ID NO: 221 - protein, 14 aa, Artificial Sequence
  Class II HLA-restricted mutated antigen FN peptide epitope
MIFEKHGFRRTTPP

SEQ ID NO: 222 - protein, 11 aa, Artificial Sequence
  Class II HLA-restricted mutated antigen Neo-PAP peptide epitope
RVIKNSIRLTL

SEQ ID NO: 223 - protein, 16 aa, Artificial Sequence
  Class II HLA-restricted mutated antigen PTPRK peptide epitope
PYYFAAELPPRNLPEP

SEQ ID NO: 224 - protein, 15 aa, Artificial Sequence
  Class II HLA-restricted mutated antigen TPI peptide epitope
GELIGILNAAKVPAD

SEQ ID NO: 225 - protein, 23 aa, Artificial Sequence
  Class II HLA-restricted mutated antigen TGFbetaRII translational frameshift peptide epitope
SLVRLSSCVPVALMSAMTTSSSQ

SEQ ID NO: 226 - protein, 11 aa, Artificial Sequence
  Class I HLA-restricted tumor-specific chimeric protein antigen abl-bcr alb-b3(b2a2) peptide epitope
FVEHDDESPGL

SEQ ID NO: 227 - protein, 10 aa, Artificial Sequence
  Class I HLA-restricted tumor-specific chimeric protein antigen abl-bcr alb-b4(b3a2) peptide epitope
FVEHDLYCTL

SEQ ID NO: 228 - protein, 9 aa, Artificial Sequence
  Class I HLA-restricted tumor-specific chimeric protein antigen bcr-abl peptide epitope
FMVELVEGA

SEQ ID NO: 229 - protein, 9 aa, Artificial Sequence
  Class I HLA-restricted tumor-specific chimeric protein antigen bcr-abl peptide epitope
KLSEQESLL

SEQ ID NO: 230 - protein, 9 aa, Artificial Sequence
  Class I HLA-restricted tumor-specific chimeric protein antigen bcr-abl peptide epitope
MLTNSCVKL

SEQ ID NO: 231 - protein, 9 aa, Artificial Sequence
  Class I HLA-restricted tumor-specific chimeric protein antigen bcr-abl p210(b3a2) peptide epitope
SSKALQRPV

SEQ ID NO: 232 - protein, 9 aa, Artificial Sequence
  Class I HLA-restricted tumor-specific chimeric protein antigen bcr-abl p210(b3a2) peptide epitope
ATGFKQSSK

SEQ ID NO: 233 - protein, 9 aa, Artificial Sequence
  Class I HLA-restricted tumor-specific chimeric protein antigen bcr-abl p210(b3a2) peptide epitope
KQSSKALQR

SEQ ID NO: 234 - protein, 11 aa, Artificial Sequence
  Class I HLA-restricted tumor-specific chimeric protein antigen bcr-abl p210(b3a2) peptide epitope
HSATGFKQSSK

SEQ ID NO: 235 - protein, 9 aa, Artificial Sequence
  Class I HLA-restricted tumor-specific chimeric protein antigen bcr-abl p210(b3a2) peptide epitope
GFKQSSKAL

SEQ ID NO: 236 - protein, 9 aa, Artificial Sequence
  Class I HLA-restricted tumor-specific chimeric protein antigen ETV6/AML peptide epitope
RIAECILGM

SEQ ID NO: 237 - protein, 10 aa, Artificial Sequence
  Class I HLA-restricted tumor-specific chimeric protein antigen NPM/ALK peptide epitope
SLAMLDLLHV

SEQ ID NO: 238 - protein, 10 aa, Artificial Sequence
  Class I HLA-restricted tumor-specific chimeric protein antigen NPM/ALK peptide epitope
GVLLWEIFSL

SEQ ID NO: 239 - protein, 10 aa, Artificial Sequence
  Class I HLA-restricted tumor-specific chimeric protein antigen SYT/SSX peptide epitope
QRPYGYDQIM

SEQ ID NO: 240 - protein, 19 aa, Artificial Sequence
  Class II HLA-restricted tumor-specific chimeric protein antigen abl-bcr alb-b3(b2a2) peptide epitope
GPHCNVFVEHDDESPGLYG

SEQ ID NO: 241 - protein, 17 aa, Artificial Sequence
  Class II HLA-restricted tumor-specific chimeric protein antigen bcr-abl p190(e1a2) peptide epitope
EGAFHGDAEALQRPVAS

SEQ ID NO: 242 - protein, 17 aa, Artificial Sequence
  Class II HLA-restricted tumor-specific chimeric protein antigen bcr-abl p210(b3a2) peptide epitope
IPLTINKEEALQRPVAS

SEQ ID NO: 243 - protein, 17 aa, Artificial Sequence
  Class II HLA-restricted tumor-specific chimeric protein antigen bcr-abl p210(b3a2) peptide epitope
ATGFKQSSKALQRPVAS

SEQ ID NO: 244 - protein, 24 aa, Artificial Sequence
  Class II HLA-restricted tumor-specific chimeric protein antigen bcr-abl p210(b3a2) peptide epitope
LIVVIVHSATGFKQSSKALQRPVA

SEQ ID NO: 245 - protein, 25 aa, Artificial Sequence
  Class II HLA-restricted tumor-specific chimeric protein antigen bcr-abl p210(b3a2) peptide epitope
IVHSATGFKQSSKALQRPVASDFEP

SEQ ID NO: 246 - protein, 16 aa, Artificial Sequence
  Class II HLA-restricted tumor-specific chimeric protein antigen DEK-CAN peptide epitope
TMKQICKKEIRRLHQY

SEQ ID NO: 247 - protein, 15 aa, Artificial Sequence
  Class II HLA-restricted tumor-specific chimeric protein antigen LDLR/FUT antisense translation peptide epitope
GGAPPVTWRRAPAPG

SEQ ID NO: 248 - protein, 15 aa, Artificial Sequence
  Class II HLA-restricted tumor-specific chimeric protein antigen LDLR/FUT antisense translation peptide epitope
WRRAPAPGAKAMAPG

SEQ ID NO: 249 - protein, 25 aa, Artificial Sequence
  Class II HLA-restricted tumor-specific chimeric protein antigen pml/RARalpha peptide epitope
NSNHVASGAGEAAIETQSSSSEEIV

SEQ ID NO: 250 - protein, 15 aa, Artificial Sequence
  Class II HLA-restricted tumor-specific chimeric protein antigen TEL/AMLI peptide epitope
IGRIAECILGMNPSR